SEQUENCE LISTING (24 sequences)

SEQ ID NO: 1 - protein, 117 aa, synthetic construct [sequence omitted]

SEQ ID NO: 2 - protein, 108 aa, synthetic construct [sequence omitted]

SEQ ID NO: 3 - protein, 5 aa, synthetic construct
  REGION 1..5: note = HCDR1
NYWIA

SEQ ID NO: 4 - protein, 17 aa, synthetic construct
  REGION 1..17: note = HCDR2
IIYPGDSDIRYSPSFQG

SEQ ID NO: 5 - protein, 8 aa, synthetic construct
  REGION 1..8: note = HCDR3
HDIEGFDY

SEQ ID NO: 6 - protein, 12 aa, synthetic construct
  REGION 1..12: note = LCDR1
RASQSVSSSFFA

SEQ ID NO: 7 - protein, 7 aa, synthetic construct
  REGION 1..7: note = LCDR2
GASSRAT

SEQ ID NO: 8 - protein, 9 aa, synthetic construct
  REGION 1..9: note = LCDR3
QQYDSSAIT

SEQ ID NO: 9 - protein, 107 aa, synthetic construct [sequence omitted]

SEQ ID NO: 10 - protein, 330 aa, synthetic construct
  REGION 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 440 aa, synthetic construct
  REGION 1..440: note = Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKS

SEQ ID NO: 12 - protein, 215 aa, synthetic construct
  REGION 1..215: note = Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, synthetic construct
  REGION 1..80: note = H15D10 (VH)
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, synthetic construct
  REGION 1..108: note = L8C3 (VL)
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, synthetic construct
  REGION 1..108: note = L16C11 (VL)
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, synthetic construct
  REGION 1..117: note = H19B7 (VH)
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

SEQ ID NO: 17 - protein, 119 aa, synthetic construct
  REGION 1..119: note = QX006N (VH)
EVQLVESGGGLVQPGGSLRLSCAASGFSLSSYYMTWVRQAPGKGLEWVSVINVYGGTYYASWAKGRFTISRDNSKNTLYLQMNSLRAEDTAVYYCAREDVAVYMAIDLWGQGTLVTVSS

SEQ ID NO: 18 - protein, 111 aa, synthetic construct
  REGION 1..111: note = QX006N (VL)
AIQMTQSPSSLSASVGDRVTITCQASQSISNQLSWYQQKPGKAPKLLIYDASSLASGVPSRFSGSRSGTKFTLTISSLQPEDFATYYCLGIYGDGADDGIAFGGGTKVEIK

SEQ ID NO: 19 - protein, 5 aa, synthetic construct
  REGION 1..5: note = QX006N (HCDR1)
SYYMT

SEQ ID NO: 20 - protein, 16 aa, synthetic construct
  REGION 1..16: note = QX006N (HCDR2)
VINVYGGTYYASWAKG

SEQ ID NO: 21 - protein, 11 aa, synthetic construct
  REGION 1..11: note = QX006N (HCDR3)
EDVAVYMAIDL

SEQ ID NO: 22 - protein, 11 aa, synthetic construct
  REGION 1..11: note = QX006N (LCDR1)
QASQSISNQLS

SEQ ID NO: 23 - protein, 7 aa, synthetic construct
  REGION 1..7: note = QX006N (LCDR2)
DASSLAS

SEQ ID NO: 24 - protein, 13 aa, synthetic construct
  REGION 1..13: note = QX006N (LCDR3)
LGIYGDGADDGIA

(19) United States
(12) Patent Application Publication
(10) Pub. No.: US 2025/0002589 A1
(43) Pub. Date: Jan. 2, 2025

(54) TREATMENT OF CUTANEOUS LUPUS ERYTHEMATOUS

(71) Applicant: ASTRAZENECA AB, SODERTALJE (SE)

(72) Inventors: KATIE STREICHER, GAITHERSBURG, MD (US); CATHARINA LINDHOLM, SODERTALJE (SE); YEN LIN CHIA, WILMINGTON, DE (US); RAJENDRA TUMMALA, WILMINGTON, DE (US); LORIN ROSKOS, GAITHERSBURG, MD (US); JOACHIM ALMQUIST, SODERTALJE (SE); TOMAS ROUSE, SODERTALJE (SE); JOAN MERRILL, WILMINGTON, DE (US); VICTORIA WERTH, WILMINGTON, DE (US)

(21) Appl. No.: 18/435,476

(22) Filed: Feb. 7, 2024

Related U.S. Application Data
(63) Continuation of application No. 18/474,601, filed on Sep. 26, 2023, now abandoned.
Application No. 18/474,601 is a continuation of application No. PCT/EP22/60669, filed on Apr. 22, 2022.
(60) Provisional application No. 63/178,750, filed on Apr. 23, 2021.

(51) Int. Cl. C07K 16/28; A61K 9/00; A61K 39/00; A61K 47/18; A61P 37/06

(52) U.S. Cl. CPC C07K 16/2866; A61K 9/0019; A61K 47/183; A61P 37/06; A61K 2039/505; A61K 2039/54; A61K 2039/545

(57) ABSTRACT

The disclosure relates to methods and compositions for the treatment of Cutaneous Lupus Erythematous (CLE). Specifically, the disclosure relates to methods comprising administering to a subject a type I IFN receptor inhibitor.

### CROSS REFERENCE TO RELATED APPLICATIONS

This present application is a continuing application U.S. application Ser. No. 18/474,601, filed Sep. 26, 2023, which is a continuing application of International Application No. PCT/EP2022/060669, filed Apr. 22, 2022, which claims priority to U.S. Provisional Application No. 63/178,750, filed Apr. 23, 2021, the entire contents of which are incorporated herein by reference in their entirety.

### REFERENCE TO AN ELECTRONIC SEQUENCE LISTING

The instant application contains an electronic Sequence Listing that has been submitted electronically and is hereby incorporated by reference in its entirety. The sequence listing was created on Jul. 26, 2023, is named “IFNAR-775_sequence_listing.xml” and is 32,768 bytes in size.

### 1. BACKGROUND

Cutaneous lupus erythematosus (CLE) is a rare, chronic, inflammatory, autoimmune, type 1 interferon (IFN)-driven, skin disease which can occur on its own, or as part of a broader diagnosis of systemic lupus erythematosus (SLE). The most commonly used treatments are topical corticosteroids, but adherence to treatment declines over time and there are side effects with continued use¹.

CLE is much more than SLE with skin involvement. CLE may be part of the spectrum of SLE or be an entity alone with no systemic features. When part of SLE, CLE may undergo flares in the absence of any other systemic exacerbation or can be part of a multiorgan flare².

Belimumab is a monoclonal antibody that reduces B lymphocyte survival by blocking the binding of soluble human B lymphocyte stimulator (BLyS) to its B cell receptors. Belimumab is approved by the FDA and EMA for the treatment of SLE but not for the treatment CLE. Indeed, no medications have been approved for CLE in over 50 years, highlighting a substantial unmet medical need for novel treatments with disease-specific mechanisms of action that reduce overall disease activity, while also reducing flares and the risk of long-term skin damage.

Anifrolumab is a human, monoclonal antibody that targets the type I IFN receptor subunit 1³. Two phase 3 randomized controlled trials (TULIP-1 and TULIP-2) have demonstrated that anifrolumab can provide therapeutic benefit across clinical endpoints and is well tolerated by patients with moderate to severe SLE. The safety and efficacy of type I IFN receptor inhibitor in patients with CLE has not previously been suggested.

The present invention solves one or more of the above-mentioned problems.

### 2. SUMMARY

The present invention relates to an improved treatment of cutaneous lupus erythematosus (CLE). The invention particularly relates to a method of treating CLE in a subject in need thereof, the method comprising administering a type I IFN receptor (IFNAR1) inhibitor to the subject, wherein the method reduces CLE disease activity in the subject. The invention is supported inter alia by data, presented herein for the first time, from two phase III, multicenter, multinational, randomized, double-blind, placebo-controlled clinical trials (NCT02446899 and NCT02962960) demonstrating that an IFNAR1 inhibitor (anifrolumab) treats rash in patients with moderate to severe CLE (i.e. having a CLASI score ≥10), in as little as 4 weeks.

The present invention further relates to a unit dose of anifrolumab suitable for use in a method of treating CLE in a subject, particular via subcutaneous administration.

### 3. BRIEF DESCRIPTION OF FIGURES

FIG. 1: IFN scores distribution.

FIG. 2A and FIG. 2B: MUSE follow-up. FIG. 2A: Patients were required to complete a 12-wk follow-up period and visits were conducted every 4 wks (±7 days) after the final study dose. FIG. 2B: IFNGS neutralization-change in neutralization ratio of the 21-gene type I IFNGS from start of the MUSE trial to the end of follow-up (week 60). From Wk 52 to Wk 60, IFNGS expression increased more rapidly in the anifrolumab 300-mg group vs the 1000-mg group.

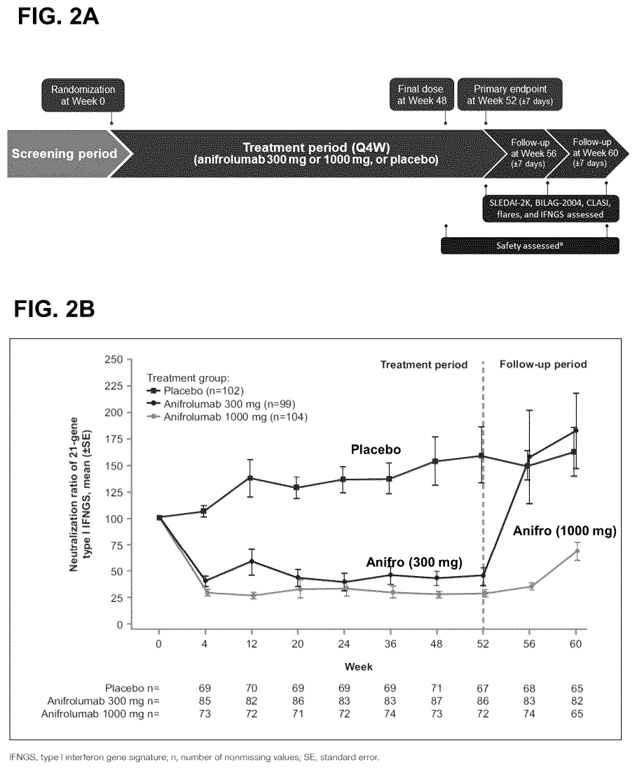

FIG. 3A and FIG. 3B: Efficacy in the MUSE trial. FIG. 3A: Disease activity measures at MUSE trial efficacy endpoint (week 52) and at end of follow-up (week 60). From Wk 52 to the end of the follow-up period (Wk 60), mean global SLEDAI-2K scores increased in patients coming off anifrolumab 300 mg and 1000 mg but not for the placebo group. A similar trend was observed in mean global BILAG-2004 scores in patients coming off anifrolumab 300 mg vs placebo. Mean CLASI scores increased slightly from Wk 52 to Wk 60 across the anifrolumab 300-mg, 1000-mg, and placebo groups. Disease activity, measured using MDGA score, increased between Week 52 and Week 60 in both anifrolumab 300-mg and 1000-mg groups; there was no change in the placebo group. Active joint counts increased slightly from Week 52 to Week 60 across the anifrolumab 300-mg, anifrolumab 1000-mg, and placebo groups. FIG. 3B: Number of flares from MUSE trial efficacy endpoint (week 52) to end of follow-up (week 60). More patients ceasing treatment of anifrolumab 300 or 1000 mg had ≥1 BILAG flare from Week 52 through Week 60 versus placebo.

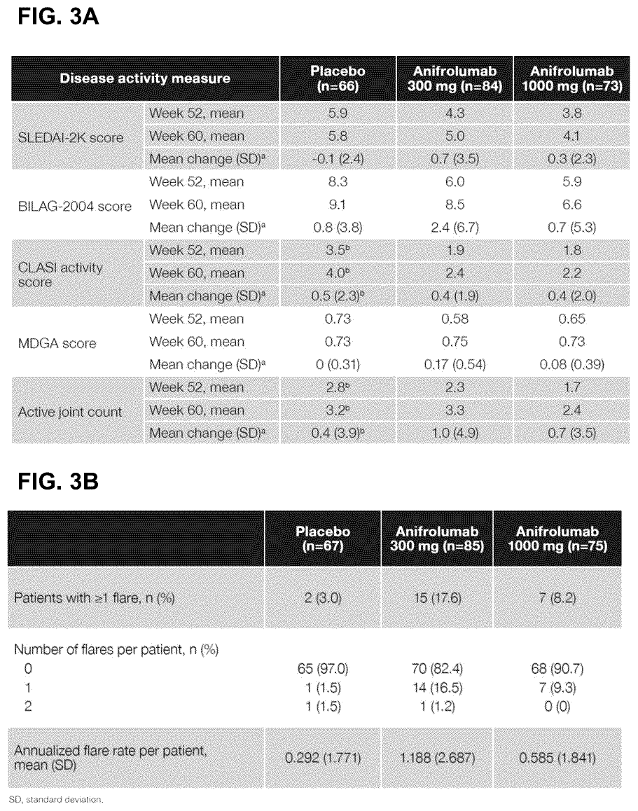

FIG. 4: Efficacy in mucocutaneous organ domain. Change in Percentages of Patients With BILAG-2004 Scores A/B and C/D/E in the Mucocutaneous Domain From MUSE Trial Efficacy Endpoint (Week 52) to End of Follow-up (Week 60). Mucocutaneous (left) was the most frequent organ system associated with worsening in patients ceasing anifrolumab, with shifts in the percentages of patients with BILAG C/D/E scores to BILAG A/B scores. Worsening was most frequent in the mucocutaneous domain in patients coming off anifrolumab, with shifts in the percentages of patients with BILAG-2004 C/D/E to A/B scores.

FIG. 5: Baseline patient demographics, disease characteristics and lupus medications. In the anifrolumab and placebo arms, the majority of patients had ≥1 BILAG A organ domain score or no A items and ≥2 B items.

FIG. 6A, FIG. 6B, and FIG. 6C: Baseline organ domain scores. Baseline (FIG. 6A) BILAG-2004 and (FIG. 6B) SLEDAI-2K organ involvement and (FIG. 6C) BILAG-2004 organ domain scores. Pb=Placebo; ANI=anifrolumab. The most commonly affected organ domains at baseline were mucocutaneous, musculoskeletal, and immunologic. Central nervous system (CNS)/neuropsychiatric and renal involvement were relatively uncommon at baseline for both BILAG-2004 and SLEDAI-2K because of the exclusion of patients with severe active lupus nephritis or severe active CNS manifestations. Baseline organ domain involvement assessed by BILAG-2004 and SLEDAI-2K was similar between treatment groups.

FIG. 7: BILAG-2004 responders at Week 52 by organ domain in TULIP-1 and TULIP-2. At Week 52, a greater number of patients treated with anifrolumab vs placebo had improvements in the BILAG-2004 mucocutaneous and musculoskeletal domain scores. Improvements were also observed in the majority of less frequently affected domains.

FIG. 8: Flares at Week 52 by Maintained OCS Dosage Reduction in Patients With Baseline OCS Dosage ≥10 mg/day in TULIP-1 and TULIP-2. BILAG, British Isles Lupus Assessment Group; OCS, oral corticosteroid; SLEDAI, SLE Disease Activity Index. Maintained OCS dosage reduction was defined as OCS dosage of ≤7.5 mg/day achieved by Week 40 and maintained to Week 52. OCS are described as “Prednisone or equivalent.” OCS administered when necessary are not considered in the calculation of the daily dose. Flares were defined as ≥1 new BILAG-2004 A or ≥2 new BILAG-2004 B domain scores versus the prior visit. Randomization in TULIP-1 and TULIP-2 was stratified by OCS dosage (<10 versus ≥10 mg/day), SLEDAI-2K score (<10 versus ≥10), and type I interferon gene signature (high versus low).

FIG. 9A, FIG. 9B, and FIG. 9C: Efficacy of anifrolumab in rash. FIG. 9A: Patients with SLEDAI-2K-defined resolution. Overall, more anifrolumab-treated patients versus placebo achieved SLEDAI-2K-defined complete resolution of rash. FIG. 9B: Patients with BIILAG-defined improvement in rash. The more sensitive measure, BILAG, which required an improvement of ≥1 grade, showed a benefit of anifrolumab over placebo for rash (difference 15.5%, nominal P<0.001); results were comparable in the IFNGS test-high subset. FIG. 9C: Patients with ≥50% improvement in mCLASI score from baseline to Week 52(mCLASI>0 at baseline). Improvements of ≥50% from baseline to Week 52, defined by mCLASI, in patients with baseline mCLASI activity scores >0, were more frequent with anifrolumab versus placebo. Data pooled from TULIP-1 and TULIP-2 trials. Resolution defined as: SLEDAI-2K rash component=0 (patients with SLEDAI-2K rash component=2 at baseline); SLEDAI-2K arthritis component=0 (patients with SLEDAI-2k arthritis component=4 at baseline); Improvement defined as: BILAG rash, mucocutaneous baseline score A change to B, C or D, or baseline score B change to C or D. mCLASI defined as the activity portions of CLASI that describe skin erythema, scale/hypertrophy, and inflammation of the scalp. BILAG, British Isles Lupus Assessment Group; IFNGS, interferon gene signature; mCLASI, modified Cutaneous Lupus Erythematosus Disease Area and Severity Index; SLEDAI-2K, Systemic Lupus Erythematosus Disease Activity Index 2000.

FIG. 10A and FIG. 10B: Baseline organ domain involvement assessed using BILAG-2004 and SLEDAI-2K. Baseline organ domain involvement assessed using BILAG-2004 (FIG. 10A) and SLEDAI-2K (FIG. 10B) was similar between treatment groups. BILAG-2004, British Isles Lupus Assessment Group-2004; SLEDAI-2K, Systemic Lupus Erythematosus Disease Activity Index 2000. BILAG-2004 scores range from level A (severe/active disease) to E (no current or previous disease). BILAG-2004 organ domain involvement was defined as an A or B score. SLEDAI-2K organ domain involvement was defined as any SLEDAI-2K organ system score. ^{a}Excluding fever.

FIG. 11: BILAG organ domain scores. BILAG organ domain scores were balanced across treatment groups. BILAG-2004 scores range from level A (severe/active disease) to E (no current or previous disease). BILAG-2004 organ domain involvement was defined as an A or B score. SLEDAI-2K organ domain involvement was defined as any SLEDAI-2K organ system score.

FIG. 12: Heat maps of individual patient BILAG-2004 mucocutaneous organ domain score over time. Patients with BILAG-2004 mucocutaneous organ domain involvement at baseline, sorted by baseline score (A or B) and Week 52 score. Each row represents an individual patient and each column represents a BILAG-2004 organ domain score every 4 weeks from Week 0 (baseline) to Week 52. Colours indicate patient BILAG-2004 scores from dark grey (A, severe/active disease) to light grey (D, no current disease).

FIG. 13: BILAG-2004 responders at Week 52 by number of score shifts. A 1-score shift is a shift from an A score at baseline to a B score at Week 52 or a B score at baseline to a C score at Week 52; a 2-score shift is from A to C or B to D; a 3-score shift is from A to D. Improvement in BILAG-2004 organ domain scores was defined as a step down from an A or B score to a B, C, or D score among patients with an A or B score at baseline. BILAG responders are the patients with improvements from baseline at Week 52. **P<0.01; ***P<0.001 (based on Cochran-Mantel-Haenszel approach for the comparison of BILAG-2004 responder rates for anifrolumab vs placebo).

FIG. 14: BILAG-2004 mucocutaneous organ domain responders over time. Improvements favoring anifrolumab for the mucocutaneous BILAG-2004 domain were observed from Week 4 and Week 32. BILAG-2004, British Isles Lupus Assessment Group-2004. BILAG-2004 organ domain responder is defined as a reduction in baseline A or B score at Week 52. Points are estimates. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

FIG. 15: SLEDAI-2K mucocutaneous organ domain responders over time. SLEDAI-2K organ domain responder is defined as a reduction in baseline SLEDAI-2K mucocutaneous organ domain score. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

FIG. 16A, FIG. 16B, and FIG. 16C. Response Rates for SLEDAI-2K-Defined Complete Resolution. (FIG. 16A), SLEDAI-2K-Defined Improvement of Rash, (FIG. 16B), BILAG-2004-Defined Improvement of Rash, and (FIG. 16C) mCLASI Improvement From Baseline ≥50% Among Patients With mCLASI>0 (C), at Week 52. BILAG-2004, British Isles Lupus Assessment Group-2004; IFNGS, interferon gene signature; mCLASI, modified Cutaneous Lupus Erythematosus Disease Area and Severity Index; SLEDAI-2K, Systemic Lupus Erythematosus Disease Activity Index 2000. Data pooled from TULIP-1 and TULIP-2 trials. Resolution defined as SLEDAI-2K rash component score of 0 in patients with SLEDAI-2K rash component score of 2 at baseline; improvement defined as BILAG rash, mucocutaneous baseline score A change to B, C, or D, or baseline score B change to C or D. mCLASI is defined as the activity portions of CLASI that describe skin erythema, scale/hypertrophy, and inflammation of the scalp.

FIG. 17A, FIG. 17B, and FIG. 17C: Response Rates for SLEDAI-2K-Defined Resolution of Rash. Response Rates for SLEDAI-2K-Defined Resolution of Rash From Baseline to Week 52 in All (FIG. 17A), IFNGS-High (FIG. 17B), and IFNGS-Low (FIG. 17C) Patients. IFNGS, interferon gene signature; SLEDAI-2K, Systemic Lupus Erythematosus Disease Activity Index 2000. Data pooled from TULIP-1 and TULIP-2 trials. Resolution defined as SLEDAI-2K rash component score of 0 in patients with SLEDAI-2K rash component score of 2 at baseline.

FIG. 18A, FIG. 18B, and FIG. 18C: Response Rates for BILAG-2004-Defined Improvement of Rash. Response Rates for BILAG-2004-Defined Improvement of Rash From Baseline to Week 52 in All (FIG. 18A), IFNGS-High (FIG. 18B), and IFNGS-Low (FIG. 18C) Patients. BILAG-2004, British Isles Lupus Assessment Group-2004; IFNGS, interferon gene signature. Data pooled from TULIP-1 and TULIP-2 trials. Improvement defined as BILAG rash, mucocutaneous baseline score A change to B, C, or D, or baseline score B change to C or D.

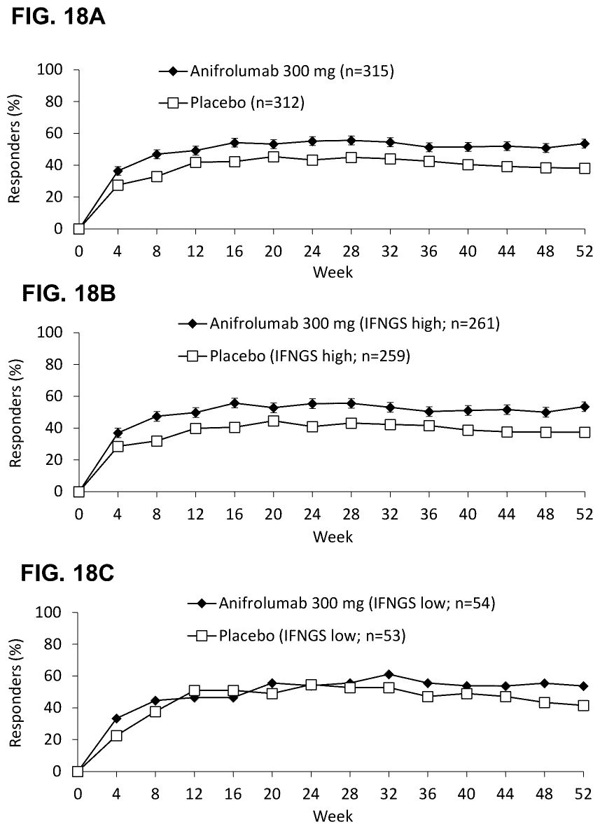

FIG. 19A, FIG. 19B, and FIG. 19C: Response Rates for ≥50% mCLASI Improvement From Baseline to Week. Response Rates for ≥50% mCLASI Improvement From Baseline to Week 52 in All (FIG. 19A), IFNGS-High (FIG. 19B), and IFNGS-Low (FIG. 19C) Patients With mCLASI>0 at Baseline.

FIG. 20: Percentages of patients who achieved ≥50% reductions from baseline CLASI-A overtime and baseline swollen joint count and tender joint count over time. CLASI response is defined as ≥50% reduction in CLASI-A from baseline for patients with baseline CLASI-A≥10. Points are estimates. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

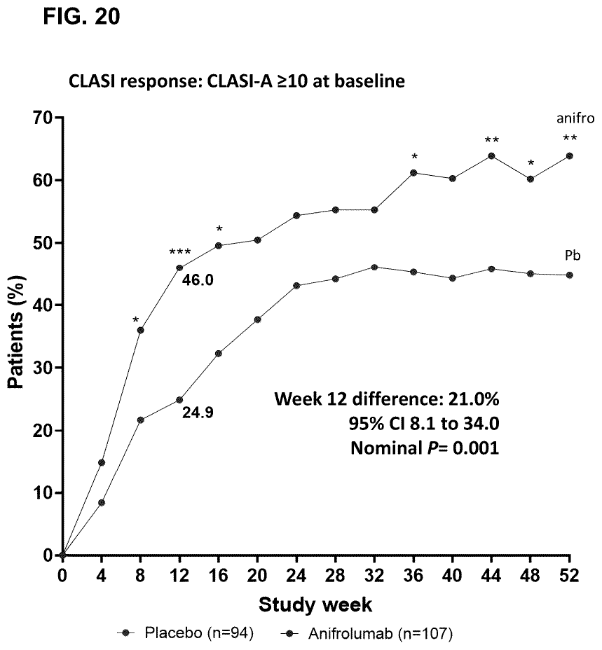

FIG. 21A and FIG. 21B: Mean anifrolumab serum concentration-time profiles. FIG. 21A: Study MI-CP180 in SSc—Mean anifrolumab serum concentration-time profiles following a single IV dose. Data represent +/−SD. Mean data below LLOQ are not plotted. IV, intravenous; LLOQ, lower limit of quantification; MEDI 546, anifrolumab; n, number of patients in a subgroup; SSc, systemic sclerosis. FIG. 21B: Study 06 in healthy volunteers—Mean anifrolumab serum concentration-time profiles following a single SC and IV dose. Samples with actual collection time deviating from nominal collection time by >10% were excluded from the mean. IV, intravenous; N, number of subjects; SC, subcutaneous.

FIG. 22A, FIG. 22B, and FIG. 22C: Study 1013 (MUSE), study design and efficacy results (20). FIG. 22A: Study design for phase II of SC anifrolumab in SLE patients. Study 08 (NCT02962960) evaluated the effect of two anifrolumab doses every other week. FIG. 22B: Mean serum concentration of anifrolumab over time. FIG. 22C: Anifrolumab neutralization of the type I IFN gene signature.

FIG. 23A and FIG. 23B: Computed median AUC Ratios (SC/IV). FIG. 23A: Computed median AUC Ratio (SC/IV) between weeks 0-52 for various SC doses. The computed median AUC Ratio (SC/IV), based on the estimated bioavailability from Study 06, between weeks 0-52, where the subcutaneous dose is either 75 mg (+ sign), 90 mg (empty squares), 105 mg (circles), 120 mg (triangles), or 135 mg (filled squares). The subcutaneous dose here is administered once every 7 days (QW); the IV dose is administered once every 4 weeks (Q4W) at a dose of 300 mg. Based on the AUC, both 90 and 105 mg SC QW appear similar to 300 mg IV. FIG. 23B: Computed median AUC ratio (SC/IV) for 90 mg and 105 mg SC QW. The computed median AUC Ratio (SC/IV), based on the estimated bioavailability ˜7% lower than the bioavailability calculated from Study 06, between weeks 0-52, where the subcutaneous dose is either 90 mg SC QW or 105 mg SC.

FIG. 24A and FIG. 24B: Anifrolumab concentration over time at different doses. FIG. 24A: A plot showing (computed) trough concentrations of plasma anifrolumab in a patient administered either (i) 105 mg of anifrolumab subcutaneously, once every 7 days (straight line); (ii) 300 mg anifrolumab intravenously, once every 4 weeks (lower dotted line); (ii) 1000 mg anifrolumab intravenously, once every 4 weeks (upper dotted line). Shaded area represents the area between 5th and 95th percentiles of the 300 mg IV Q4W dose. FIG. 24B: Anifrolumab trough concentration in IFNGS high SLE subjects. Computed trough concentrations of anifrolumab in IFNGS high patients plasma after administration as follows: (i) 300mg IV Q4W; (ii) 90 mg SC QW; (iii) 105 mg SC QW; (iv) 135 mg SC QW; (v) 1000 mg IV Q4W. SC=subcutaneous. Based on trough, both 90 and 105 mg SC QW were projected to have higher PD suppressions than 300 mg IV.

FIG. 25A and FIG. 25B: Positive Exposure-BICLA relationship observed in TULIP 1 & TULIP 2 in IFNGS high patients. FIG. 25A: TULIP I, for placebo, 150 mg and 300 mg anifrolumab. FIG. 25B: TULIP II, for placebo and 300 mg.

FIG. 26A and FIG. 26B: BICLA dose response. FIG. 26A: Dose response curve, for probability of meeting BICLA response criteria (in IFNGS high patients) versus anifrolumab Cave over 52 weeks, showing the predicted mean (grey line) and 95% confidence interval (CI) (dashed area). Patients are grouped by dose (150 mg, n=62; 300 mg, n=242; and 1000 mg). FIG. 26B: Predicted PK and efficacy for different SC doses. The probability of meeting BICLA (in IFNGS high patients) for weekly subcutaneous doses starting from 105 mg, and up to 150 mg. Assumptions for generating the data include no dose delays/interruptions.

FIG. 27A and FIG. 27B: C_{troughs} following injection at thigh compared to injection at abdomen. FIG. 27A: 150 mg SC Q2W. FIG. 27B: 300 mg SC Q2W.

FIG. 28: Exposure prediction based on 81-87% bioavailability and preliminary PK modelling. Anifrolumab Cₐᵥₑ medium ratio predicted for 90-150 mg SC QW to 300 mg Q4W, based on PK preliminary modelling and bioavailability assumptions.

FIG. 29A, FIG. 29B, and FIG. 29C: Anifrolumab Cₐᵥₑ over 52 weeks in IFNGS high patients for different SC and IV doses. FIG. 29A: 105 mg SC QW. FIG. 29B: 120 mg SC QW. FIG. 29C: Overlap with 1000 mg IV Q4W.

FIG. 30A and FIG. 30B: Cₐᵥₑ median ratio SC QW to 300 mg IV Q4W. FIG. 30A: 81% bioavailability assumed. FIG. 30B: 70% bioavailability assumed.

FIG. 31: Average anifrolumab concentration versus herpes zoster incidence. The incidence of Herpes Zoster (%) in patients in the Study 1013 receiving placebo, 300 mg IV anifrolumab or 1000 mg IV anifrolumab.

FIG. 32A and FIG. 32B. Delivery device. Anifrolumab is administered by an injection device [1] [9] such as a prefilled syringe (PFS) (FIG. 32A) or an autoinjector (AI) (FIG. 32B).

FIG. 33A, FIG. 33B, and FIG. 33C. Autoinjector. The autoinjector for administering anifrolumab of the functional variant thereof in exploded view (FIG. 33A), assembled (FIG. 33B) and filled with drug substance (FIG. 33C).

FIG. 34A, FIG. 34B, FIG. 34C, and FIG. 34D. Accessorized pre-filled syringe. The accessorized pre-filled syringe (APFS) for anifrolumab of the functional variant thereof. The primary tube is shown in assembled form (FIG. 34A) and in exploded view (FIG. 34B). The APFS with its additional components is shown in assembled form (FIG. 34C) and in exploded view (FIG. 34D).

FIG. 35. Packaging for the delivery device

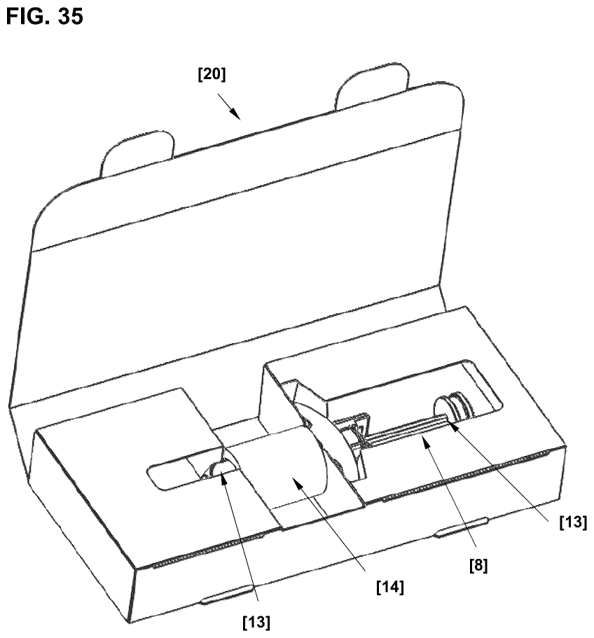

### 4. DETAILED DESCRIPTION

### 4.1. Method of Treating Cutaneous Lupus Erythematous (CLE)

The invention relates to a method of treating cutaneous lupus erythematous (CLE) in a subject in need thereof, the method comprising administering a type I IFN receptor (IFNAR1) inhibitor to the subject, wherein the method reduces CLE disease activity in the subject.

Reducing CLE disease activity in the subject may comprise reducing rash in the patient compared to pretreatment. Reducing CLE disease activity in the subject may comprise resolving rash in the patient. Reducing CLE disease activity may comprise ≥50% improvement in rash compared to pretreatment. Reducing CLE disease activity may comprise ≥50% improvement in rash compared to pretreatment as assessed by mCLASI. Pretreatment, the subject may have a CLASI or mCLASI score of ≥6 or ≥10 pretreatment. The reduction in CLE activity may be achieved by week 4 of treatment. The IFNAR1 inhibitor may reduce interferon stimulated gene expression in the skin.

### 4.2. IFNAR1 Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably a interferon-a/B receptor (IFNAR). IFNAR1 is a subunit of the IFNAR. IFNAR1 and IFNAR are used herein interchangeably.

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain variable region complementarity determining region 2 (HCDR2) comprising the amino acid sequence of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12.

The IFNAR1 inhibitor may be anifrolumab or a functional variant thereof.

### 4.3. Doses and Methods of Administration

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The subject may have moderate to severe CLE pre-treatment. The subject may have mild CLE. Moderate to severe CLE may be defined as a CLASI score of ≥10.

The subject may be a type I interferon stimulated gene signature (IFNGS)-test high patient pre-treatment. The method may comprise identifying the subject as IFNGS-test high patient pre-treatment.

Many patients with CLE receive corticosteroids (glucocorticoids, oral corticosteroids, OCS). However, corticosteroids are associated with organ damage. Anifrolumab permits tapering of the corticosteroids (glucocorticoids) in CLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

The invention also relates to a unit dose for use in the methods of the invention, wherein the unit dose comprises >105 mg and ≤150 mg an IFNAR inhibitor (e.g. anifrolumab or a functional variant thereof). The unit dose may comprise 105 to 149 mg of an IFNAR inhibitor.

The unit dose may comprise ≤135 mg (i.e. 135 mg or less) an IFNAR inhibitor (e.g. anifrolumab or the functional variant thereof). The unit dose may comprise 105 mg to 135 mg of an IFNAR inhibitor. The unit dose may comprise about 120 mg of an IFNAR inhibitor (e.g. anifrolumab or the functional variant thereof). The unit dose may comprise 120 mg of an IFNAR inhibitor (e.g. anifrolumab or the functional variant thereof). The unit dose may consist essentially of >105 mg and <150 mg of an IFNAR inhibitor (anifrolumab or the functional variant thereof). The unit dose may consist essentially of ≤135 mg of an IFNAR inhibitor (e.g. anifrolumab or the functional variant thereof). The unit dose may consist essentially of about 120 mg of an IFNAR inhibitor (e.g. anifrolumab or the functional variant thereof). The concentration of the IFNAR inhibitor (e.g. anifrolumab or the functional variant thereof) in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine salt and an uncharged excipient. The unit dose may comprise a formulation of 150 to 200 mg/ml anifrolumab or the functional variant thereof, 25 to 150 mM of lysine salt and an uncharged excipient. The unit dose comprises a formulation of 25 mM histidine-HCL, 130 mM trehalose, and 0.05% w/v polysorbate 80. The formulation may have a pH of about 5.9.

In another aspect the invention relates to a method of treating CLE in a subject, the method comprising subcutaneously administering a dose of anifrolumab or a functional variant thereof, wherein administering the dose every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is more than the plasma concentration provided by intravenous administration of 300 mg of anifrolumab or the functional variant thereof every 4 weeks. Administering the dose every week may provide a plasma concentration in the subject that is at least equivalent to the plasma concentration provided by intravenous administration of 400 mg of anifrolumab or the functional variant thereof every 4 weeks. The dose may be administered in a single-administration step. The dose administered to the subject may be <150 mg (i.e. less than 150 mg) anifrolumab or the functional variant thereof. The dose administered to the subject may be >105 mg (i.e. more than 105 mg) anifrolumab or the functional variant thereof. The dose of administered to the subject may be ≤135 mg (i.e. 135 mg or less) anifrolumab or the functional variant thereof. The dose administered to the subject may be about 120 mg anifrolumab or the functional variant thereof.

Administration of the dose or unit dose may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 μg/ml. Administration of the dose or unit dose may provide a plasma concentration of anifrolumab or the functional variant thereof in the subject of about 30-70 μg/ml. Administration of the dose or unit dose may provide a trough concentration of anifrolumab or the functional variant thereof in the subject of ≥20 μg/ml (i.e. 20 μg/ml or more). Administration of the dose or unit dose may provide a trough concentration of anifrolumab or the functional variant thereof in the subject of ≥30 μg/ml (i.e. 30 μg/ml or more). Administration of the dose or unit dose may provide a trough concentration of anifrolumab or the functional variant thereof in the subject of ≥40 μg/ml (i.e. 40 μg/ml or more). Administration of the dose or unit dose may provide a trough concentration of anifrolumab or the functional variant thereof in the subject of about 20-100 μg/ml. Administration of the dose or unit dose may provide a trough concentration of anifrolumab or the functional variant thereof in the subject of about 30-80 μg/ml. Administration of the dose or unit dose may provide a trough concentration of anifrolumab or the functional variant thereof in the subject of about 40-70 μg/ml.

In another aspect the invention relates to a method of treating a CLE in a subject, the method comprising subcutaneously administering a dose of anifrolumab or a functional variant thereof, wherein the dose 105 mg to 149 mg.

The dose or unit dose may be 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg or 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, or 149 mg.

### 4.4. The Subject

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The dose or unit dose may provide a therapeutic effect in the subject that is at least equivalent to a therapeutic effect provided by administration of an intravenous dose of 300 mg anifrolumab or the functional variant thereof administered once every (Q4W). The dose or unit dose may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine salt and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The methods of the invention may comprise administering the dose or unit dose at intervals of 6-8 days. The dose or unit dose may be administered once per week (QW). The dose or unit dose may be 120 mg anifrolumab or the functional variant thereof, wherein the method comprises administering the dose in a single administration step once per week (QW). In other words, the method comprises administering 120 mg QW of anifrolumab of the functional variant thereof. The dose or unit dose may be administered once per week for at least about 4 weeks. The dose or unit dose may be administered once per week for at least about 8 weeks. The dose or unit dose may be administered once per week for at least about 12 weeks. The dose or unit dose may be administered once per week for at least about 16 weeks. The dose or unit dose may be administered once per week for at least about 20 weeks. The dose or unit dose may be administered once per week for at least about 24 weeks. The dose or unit dose may be administered once per week for at least about 28 weeks. The dose or unit dose may be administered once per week for at least about 32 weeks. The dose or unit dose may be administered once per week for about 8 weeks. The dose or unit dose may have a volume permitted it suitable delivery in a single subcutaneous administration step. The dose or unit dose may have a volume of about 0.5 to about 1 ml. The dose or unit dose may have a volume of less than 1 ml. The dose or unit dose may have a volume of about 0.8 ml.

### 4.5. Pharmaceutical Composition

The invention also relates to a pharmaceutical composition for use in a method of treating CLE in a subject, the method comprising subcutaneously administering the pharmaceutical composition to a subject, wherein the pharmaceutical composition comprises a dose of anifrolumab or functional variant thereof, wherein the dose is >105 mg and <150 mg. The dose of anifrolumab of the functional variant thereof may be a unit dose (unit dose form, pharmaceutical unit dose form, pharmaceutical unit dose). Functional anifrolumab variants include antigen-binding fragments of anifrolumab and antibody and immunoglobulin derivatives of anifrolumab.

In another aspect the invention relates to a pharmaceutical composition for use in a method of treating CLE in a subject, the method comprising subcutaneously administering the pharmaceutical composition to the subject, wherein the pharmaceutical composition comprises a dose of anifrolumab or functional variant thereof, wherein administering the pharmaceutical composition every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is about equivalent to the plasma concentration provided by intravenous administration of 400 mg of anifrolumab or the functional variant thereof every 4 weeks. The dose may be <150 mg (i.e. less than 150 mg) anifrolumab or the functional variant thereof. The dose may be >105 mg (i.e. more than 105 mg) anifrolumab or the functional variant thereof. The dose may be ≤135 mg (i.e. 135 mg or less) anifrolumab or the functional variant thereof. The dose may be about 120 mg anifrolumab or the functional variant thereof. The dose may be 120 mg anifrolumab or the functional variant thereof.

The pharmaceutical composition may be administered at intervals of 6-8 days. The pharmaceutical composition may be administered once per week (QW). The pharmaceutical composition may be administered in a single administration step. The dose may be 120 mg anifrolumab or the functional variant thereof, and the method of treatment may comprise administering the dose in a single administration step once per week (QW). The pharmaceutical composition may be administered once per week for at least about 4 weeks. The pharmaceutical composition may be administered once per week for at least about 8 weeks. The dose or unit dose may be administered once per week for at least about 12 weeks. The pharmaceutical composition may be administered once per week for at least about 16 weeks. The pharmaceutical composition may be administered once per week for at least about 20 weeks. The pharmaceutical composition may be administered once per week for at least about 24 weeks. The pharmaceutical composition may be administered once per week for at least about 28 weeks. The pharmaceutical composition may be administered once per week for at least about 32 weeks. The pharmaceutical composition may be administered once per week for about 8 weeks. The pharmaceutical composition may have a volume permitted it suitable delivery in a single subcutaneous administration step. The pharmaceutical composition may have a volume of about 0.5 to about 1 ml. The pharmaceutical composition may have a volume of less than 1 ml. The pharmaceutical composition may have a volume of about 0.8 ml.

Administration of the pharmaceutical composition may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the pharmaceutical composition may provide a plasma concentration of anifrolumab or the functional variant thereof in the subject of about 10-100 μg/ml. Administration of the pharmaceutical composition may provide a plasma concentration of anifrolumab or the functional variant thereof in the subject of about 20-80 μg/ml. Administration of the pharmaceutical composition may provide a plasma concentration of anifrolumab or the functional variant thereof in the subject of about 30-70 μg/ml. Administration of the pharmaceutical composition may provide a trough concentration of anifrolumab or the functional variant thereof in the subject of ≥20 μg/ml (i.e. 20 μg/ml or more). Administration of the pharmaceutical composition may provide a trough concentration of anifrolumab or the functional variant thereof in the subject of ≥30 μg/ml (i.e. 30 μg/ml or more). Administration of the pharmaceutical composition may provide a trough concentration of anifrolumab or the functional variant thereof in the subject of ≥40 μg/ml (i.e. 40 μg/ml or more). Administration of the pharmaceutical composition may provide a trough concentration of anifrolumab or the functional variant thereof in the subject of about 20-100 μg/ml. Administration of the pharmaceutical composition may provide a trough concentration of anifrolumab or the functional variant thereof in the subject of about 30-80 μg/ml. Administration of the pharmaceutical composition may provide a trough concentration of anifrolumab or the functional variant thereof in the subject of about 40-70 μg/ml.

The pharmaceutical composition may provide a therapeutic effect in the subject that is at least equivalent to a therapeutic effect provided by administration of an intravenous dose of 300 mg anifrolumab or the functional variant thereof administered once every (Q4W). The pharmaceutical composition may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine salt and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The pharmaceutical composition may comprise about 150 to 200 mg/ml anifrolumab or the functional variant thereof, about 25 to 150 mM of lysine salt and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise about 150 to 200 mg/ml anifrolumab or the functional variant thereof, about 25 to 150 mM of lysine salt and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

### 4.6. Device

The invention also relates to an injection device comprising the unit dose of the invention, or the pharmaceutical composition for the use of any of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine salt and an uncharged excipient. The pharmaceutical composition in the injection device may comprise 150 mg/mL anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine salt and an uncharged excipient. The pharmaceutical composition in the injection device may comprise 150 mg/mL anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose. The unit dose may comprise >105 mg (i.e. at least 105 mg) and <150 mg (i.e. less than 150 mg) anifrolumab or a functional variant thereof. The unit dose may comprise ≤135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine salt and an uncharged excipient. The unit dose in the injection device may comprise a formulation of 150 to 200 mg/ml anifrolumab or the functional variant thereof, 25 to 150 mM of lysine salt and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (Al).

### 4.7. Kit

In another aspect the invention relates to a kit comprising a unit dose of the invention and instructions for use, wherein the instructions for use comprise instructions for subcutaneous administration of the unit dose to a subject.

In another aspect the invention relates to a kit comprising the pharmaceutical composition for the use of the invention, wherein the instructions for use comprise instructions for subcutaneous administration of the pharmaceutical composition to a subject.

In another aspect the invention relates to a kit comprising the injection device of any of the invention, and instructions for use, wherein the instruction for use comprise instructions for use of the injection device to subcutaneously administer the unit dose or pharmaceutical composition to the subject.

The kit of the invention may comprise packaging, wherein the packaging is adapted to hold the injection device and the instructions for use. The instructions for use may be attached to the injection device. The instruction for use may comprise instructions for administration of >105 mg and <150 mg anifrolumab or functional variant thereof. The instruction for use may comprise instructions for administration of ≤135 mg anifrolumab or the functional variant thereof. The instruction for use may comprise instructions for administration of 120 mg anifrolumab or the functional variant thereof. The instruction for use may comprise instructions for administration of 120 mg anifrolumab or the functional variant thereof every 4 weeks. The instructions for use may define the subject as having a type I IFN mediated disease. The instructions may define the subject as having CLE. The instructions may define the subject as having moderate to severe CLE. The instructions for use may be written instructions.

The instructions for use may specify that the injection device, unit dose and/or pharmaceutical composition are for use in the treatment of CLE. The instructions for use comprise instructions for administration of 120 mg anifrolumab or the functional variant thereof every week.

### 4.8. Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine salt and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. Pat. No. 10,125,195 B1, which is incorporated herein in its in entirety.

### 5. DEFINITIONS

### 5.1. Anifrolumab

Anifrolumab (MEDI-546, anifro, ANI) is a human immunoglobulin G1 kappa (IgG1κ) monoclonal antibody (mAb) directed against subunit 1 of the type I interferon receptor (IFNAR1). Anifrolumab downregulates IFNAR signaling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Table 5-1: Sequences.

TABLE 5-1
Sequences

| Description | SEQ ID | Sequence |
|---|---|---|
| Anifrolumab VH | 1 | EVQLVQSGAEVKKPGESLKISCKGSGYIFTNYWIAWVRQMPGKGLESMGIIYPGDS |
| | | DIRYSPSFQGQVTISADKSITTAYLQWSSLKASDTAMYYCARHDIEGFDYWGRGTL |
| | | VTVSS |
| Anifrolumab VL | 2 | EIVLTQSPGTLSLSPGERATLSCRASQSVSSSFFAWYQQKPGQAPRLLIYGASSRA |
| | | TGIPDRLSGSGSGTDFTLTITRLEPEDFAVYYCQQYDSSAITFGQGTRLEIK |
| HCDR1 | 3 | NYWIA |
| HCDR2 | 4 | IIYPGDSDIRYSPSFQG |
| HCDR3 | 5 | HDIEGFDY |
| LCDR1 | 6 | RASQSVSSSFFA |
| LCDR2 | 7 | GASSRAT |
| LCDR3 | 8 | QQYDSSAIT |
| Light chain constant | 9 | RTVAAPSVFIFPPSDEQLKSGTASVVCLINNFYPREAKVQWKVDNALQSGNSQESV |
| region | | TEQDSKDSTYSLSSTLTLSKADYEKHKVYACEVTHQGLSSPVTKSFNRGEC |
| Heavy chain | 10 | ASTKGPSVFPLAPSSKSTSGGTAALGCLVKDYFPEPVTVSWNSGALTSGVHTFPAV |
| constant region | | LQSSGLYSLSSVVTVPSSSLGTQTYICNVNHKPSNTKVDKRVEPKSCDKTHTCPPC |
| | | PAPEFEGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPASIEKTISKAKG |
| | | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| Heavy chain | 11 | EVQLVQSGAEVKKPGESLKISCKGSGYIFTNYWIAWVRQMPGKGLESMGIIYPGDS |
| | | DIRYSPSFQGQVTISADKSITTAYLQWSSLKASD TAMYYCARHD |
| | | IEGFDYWGRGTLVTVSSASTKGPSVFPLAPSSKSTSGGTAALGCLVKDYFPEPVTV |
| | | SWNSGALTSGVHTFPAVLQSSGLYSLSSVVTVPS SSLGTQTYIC |
| | | NVNHKPSNTKVDKRVEPKSCDKTHTCPPCPAPEFEGGPSVFLFPPKPKDTLMISRT |
| | | PEVTCVVVDVSHEDPEVKFNWYVDGVEVHNAKTK |
| | | PREEQYNSTYRVVSVLTVLHQDWLNGKEY KCKVSNKALPA SIEKTISKAK |
| | | GQPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKS LSLSPGK |
| Light chain | 12 | EIVLTQSPGTLSLSPGERATLSCRASQSVS SSFFAWYQQK PGQAPRLLIY |
| | | GASSRATGIPDRLSGSGSGT DFTLTITRLE PEDFAVYYCQ QYDSSAITFG |
| | | QGTRLEIKRTVAAPSVFIFPPSDEQLKSGT ASVVCLLNNF YPREAKVQWK |
| | | VDNALQSGNSQESVTEQDSKDSTYSLSSTLTLSKADYEKHKVYACEVTHQGLSSPV |
| | | TKSFNRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and preferably with the same affinity.

Functional variants of anifrolumab include the antibodies described in WO 2018/023976 A1, incorporated herein by reference (Table 5-2).

TABLE 5-2
anti-IFNAR antibody sequences

| Description | SEQ ID | Sequence |
|---|---|---|
| H15D10 (VH) | 13 | EVQLVQSGAEVKKPGESLRISCKGSGYTFTNYWVAWVRQMPGKGLESMG |
| | | IIYPGDSDTRYSPSFQGHVTISADKSISTAY |
| L8C3 (VL) | 14 | DIQMTQSPSSLSASLGDRVTITCRASQNVGNYLNWYQQKPGKAPKLLIY |
| | | RASNLASGVPSRFSGSGSGTDFTLTISSLQPEDFATYYCQQMEHAPPTF |
| | | GQGTKVEIKR |
| L16C11 (VL) | 15 | EIVLTQSPGTLSLSPGERATLSCRASQSVIGYYLAWYQQKPGQAPRLLI |
| | | YSVSTLASGIPDRFSGSGSGTDFTLTISRLEPEDFAVYYCQQYYRFPIT |
| | | FGQGTKVEIK |
| H19B7 (VH) | 16 | EVQLVQSGAEVKKPGESLRISCKGSGYTFTNYWMAWVRQMPGKGLESMG |
| | | IIYPSDSDTRYSPSFQGHVTISADKSISTAYLQWSSLKASDTAMYYCAR |
| | | HDVEGYDYWGQGTLVTVSS |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VH amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

Functional variants of anifrolumab and anti-IFNAR antibodies include the QX006N antibody described in CN 11327807, incorporated herein by reference. Functional variants of anifrolumab include the antibodies described in WO 2018/023976 A1, incorporated herein by reference (Table 3).

TABLE 3
QX006N antibody sequences

| Description | SEQ ID NO | Sequence |
|---|---|---|
| QX006N (VH) | 17 | EVQLVESGGGLVQPGGSLRLSCAASGFSLSSYYMTWVRQAPGKGLEW |
| | | VSVINVYGGTYYASWAKGRFTISRDNSKNTLYLQMNSLRAEDTAVYY |
| | | CAREDVAVYMAIDLWGQGTLVTVSS |
| QX006N (VL) | 18 | AIQMTQSPSSLSASVGDRVTITCQASQSISNQLSWYQQKPGKAPKLL |
| | | IYDASSLASGVPSRFSGSRSGTKFTLTISSLQPEDFATYYCLGIYGD |
| | | GADDGIAFGGGTKVEIK |
| QX006N (HCDR1) | 19 | SYYMT |
| QX006N (HCDR2) | 20 | VINVYGGTYYASWAKG |
| QX006N (HCDR3) | 21 | EDVAVYMAIDL |
| QX006N (LCDR1) | 22 | QASQSISNQLS |
| QX006N (LCDR2) | 23 | DASSLAS |
| QX006N (LCDR3) | 24 | LGIYGDGADDGIA |

IFNAR inhibitors may be a monoclonal antibody comprising the VH amino acid sequence SEQ ID NO: 17. The anti-IFNAR antibodies may comprise the VL amino acid sequence SEQ ID NO: 18.

QX006N is an immunoglobulin comprising an HCDR1, HCDR2 and HCDR3 of SEQ ID NO: 19, SEQ ID NO: 20, and SEQ ID NO: 21, respectively (or functional variant thereof); and an LCDR1, LCDR2 and LCDR3 of SEQ ID NO: 22, SEQ ID NO: 23, and SEQ ID NO: 23, respectively (or functional variant thereof). QX006N is an immunoglobulin comprising a VH amino acid sequence SEQ ID NO: 17 the VL amino acid sequence SEQ ID NO: 18.

### 5.2. Cutaneous Lupus Erythematous

Cutaneous lupus erythematosus (CLE) is a rare, chronic, inflammatory, autoimmune, type 1 interferon (IFN)-driven, skin disease which can occur on its own, or as part of a broader diagnosis of systemic lupus erythematosus (SLE). There are 3 subtypes of CLE: acute CLE, subacute CLE, and chronic CLE. Chronic CLE is the most diverse category and is further categorized into discoid CLE, chilblain LE, LE panniculitis, and tumid lupus (also called intermittent CLE, and sometimes classed as a separate subtype Common trigger factors that aggravate CLE are UV exposure, smoking, and certain medications. Infections and hormones may also trigger CLE symptoms. No therapies are currently licensed specifically to treat CLE, highlighting a substantial unmet medical need for novel treatments with disease-specific mechanisms of action that reduce overall disease activity, while also reducing flares and the risk of long- term skin damage. The most commonly used treatments are topical corticosteroids, but adherence to treatment declines over time and there are side effects with continued use^{1,6}. Other ‘off-label’ treatments include systemic treatment with antimalarials (hydroxychloroquine), steroids, methotrexate, azathioprine, mycophenolate sodium, mycophenolate mofetil, dapsone, thalidomide and lenalidomide, and local/topical treatment with triamcinolone (intralesional injections), calcineurin inhibitors, RSalbutamol, retinoids, R333, clindamycin and Janus kinase inhibitors¹. Preventative treatments include UV protection, smoking cessation, elimination of photosensitizing drugs, and vitamin D supplementation¹.

### 5.3. Steroids

Oral corticosteroids (OCS, glucocorticoids) include prednisone, cortisone, hydrocortisone, methylprednisolone, prednisolone and triamcinolone. Examples of equivalent doses of oral prednisone are shown in Table 5-4.

TABLE 5-4
Examples of equivalent doses of oral prednisone

| Oral Prednisone and | | | | | |
|---|---|---|---|---|---|
| Equivalents | Equivalent Dose | | | | |
| Oral Prednisone | 7.5 mg | 10 mg | 20 mg | 30 mg | 40 mg |
| Cortisone | 37.5 mg | 50 mg | 100 mg | 150 mg | 200 mg |
| Hydrocortisone | 30 mg | 40 mg | 80 mg | 120 mg | 160 mg |
| Methylprednisolone | 6 mg | 8 mg | 16 mg | 24 mg | 32 mg |
| Prednisolone | 7.5 mg | 10 mg | 20 mg | 30 mg | 40 mg |
| Triamcinolone | 6 mg | 8 mg | 16 mg | 24 mg | 32 mg |

### 5.4. Dosage Forms

A unit dose (also referred to as a unit dose form, a pharmaceutical unit dose or a pharmaceutical unit dose form) is a dose formed from a single unit. A unit dose (unit dose form) is suitable for administration to a subject in a single administration step. A unit dose (unit dose form) may be packaged in a single-unit container, for example a single-use pre-filled syringe or autoinjector. Unit doses provide the advantage that they can be ordered, packaged, handled and administered as single dose units containing a pre-determined amount of a drug. Unit doses decrease administration errors and reduce waste.

### 5.5. PK/PD

Plasma levels obtainable by SC administration and IV administration may be compared on the basis of a plasma drug concentration-time curve (AUC), which reflects the body exposure to the antibody after administration of a dose of the drug. For example, during a clinical study, the patient's plasma drug concentration-time profile can be plotted by measuring the plasma concentration at several time points. Where an in silico modelling approach is employed, plasma drug concentration-time for any given dose may be predicted. The AUC (area under the curve) can then be calculated by integration of the plasma drug concentration-time curve. Suitable methodology is described in Tummala et. al.⁴, which is incorporated herein by reference in its entirety. In the Examples described herein, PK parameters were calculated by non-compartmental analysis with Phoenix WinNonlin V/6.2 (Certara, Inc., Princeton, New Jersey, USA) and included the area under the serum concentration-time curve (AUC), clearance (CL, CL/F), maximum serum concentration (Cₘₐₓ) and time to reach maximum serum concentration (tₘₐₓ). All data were analysed with SAS System V.9.2 (SAS Institute, Inc., Cary, NC, USA).

Conveniently, a ratio of the AUC obtainable with SC administration to the AUC obtainable by IV administration (AUC_{SC}/AUC_{IV}) may be calculated, providing a numerical comparison of bioavailability provided by the dosage routes. Reference to the “AUC Ratio” herein means the AUC_{SC}/AUC_{IV} ratio. To provide statistical robustness, the AUC ratio is preferably a mean, median or mode (for example, a mean) value calculated from a plurality of repeat experiments (or computational simulations). This approach is demonstrated with reference to the Examples. The mean, median or mode (preferably mean) may be derived by pooling data obtained from multiple patients (or multiple computational simulations). Thus, the AUC Ratio may reflect the mean, median or mode (preferably mean) AUC in multiple patients.

### 5.6. Pharmacokinetics Glossary

Area under the curve (AUC): Area under the plasma drug concentration versus time curve, which serves as a measure of drug exposure.

Cₐᵥₑ: Steady-state average concentration.

Cₘₐₓ: The maximum (or peak) concentration of the drug in the plasma.

Cₘᵢₙ: Minimum plasma drug concentration.

C_{trough}: the concentration of drug in plasma at steady state immediately prior to the administration of a next dose. Trough plasma concentration (measured concentration at the end of a dosing interval at steady state [taken directly before next administration]).

LLOQ: The lower limit of quantitation, the lowest amount of an analyte in a sample that can be quantitatively determined with suitable precision and accuracy.

Linear pharmacokinetics: When the concentration of the drug in the blood or plasma increases proportionally with the increasing dose, and the rate of elimination is proportional to the concentration, the drug is said to exhibit linear pharmacokinetics. The clearance and volume of distribution of these drugs are dose-independent.

Nonlinear pharmacokinetics: As opposed to linear pharmacokinetics, the concentration of the drug in the blood or plasma does not increase proportionally with the increasing dose. The clearance and volume of distribution of these may vary depending on the administered dose. Nonlinearity may be associated with any component of the absorption, distribution, and/or elimination processes.

### 5.7. Delivery Device

As well as providing for subcutaneous administration of the antibody, the ability to self-administer (e.g. for home use) may further be enhanced by subcutaneous administration via an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al.⁵, which is incorporated herein by reference in its entirety.

The dose elucidated by the inventors provides yet advantages in the context of APFS-administration, as an APFS device typically administers a maximal volume of 1 ml. A dose in the range of >105 mg to <155 mg can be readily accommodated by a volume of ˜0.8 ml, such that the dose(s) of the present invention are uniquely suited to APFS and AI administration. For comparison, due to viscosity of the anifrolumab, larger doses (particularly doses of >150 mg) would need to be administered within a volume of >1 ml, requiring at least two SC injections, which is inconvenient for the patient, and would require a plurality of pre-filled devices.

The delivery device may be single use, disposable system that is designed to enable manual, SC administration of the dose.

### 5.8. End Points

### 5.8.1. BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorises disease activity into 5 different levels from A to E:
Grade A represents very active disease requiring immunosuppressive drugs and/or a prednisone dose of >20 mg/day or equivalent
Grade B represents moderate disease activity requiring a lower dose of corticosteroids, topical steroids, topical immunosuppressives, antimalarials, or NSAIDs
Grade C indicates mild stable disease
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

BILAG-defined improvement in mucocutaneous or musculoskeletal organ systems were representative of rash or arthritis, respectively.

### 5.8.2. BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of >0 points in SLEDAI-2K;
c) No worsening from baseline in the subjects' lupus disease activity defined by an increase ≥0.30 points on a 3-point PGA VAS;
d) No discontinuation of investigational product or use of restricted medications beyond the protocol-allowed threshold before assessment

### 5.8.3. CLASI (Cutaneous Lupus Erythematosus Disease Area and Severity Index Inflammatory Disease Activity)

The Cutaneous Lupus Erythematosus Disease Area and Severity Index (CLASI) was developed in 2005 as a means of specifically tracking cutaneous activity and damage in patients with CLE⁷. The CLASI is a simple, single-page tool that separately quantifies skin disease activity and damage in each part of the body⁸. The CLASI features a skin activity summary score (CLASI-A) and damage summary score (CLASI-D). This index has a high inter-rater and intra-rater reliability and is responsive to change when used in adults with CLE and SLE. CLASI activity score correlates with the severity of disease: mild, moderate, and severe disease corresponded with CLASI activity score ranges of 0-9 (sensitivity 93%, specificity 78%), 10-20, and 21-70 (sensitivity 80%, specificity 95%), respectively (Table 5-5).

TABLE 5-5
CLE disease severity based on the CLASI activity score

| CLE disease | CLASI activity score range |
|---|---|
| Mild | 0-9 |
| Moderate | 10-20 |
| Severe | 21-70 |

The Cutaneous Lupus Erythematosus Disease Area and Severity Index (CLASI) quantifies disease activity and damage in cutaneous lupus erythematosus. It can distinguish between different response levels of treatment, e.g., it is able to detect a specific percentage reduction in activity score from baseline, or can be reported by a mean/median score. Particularly, the CLASI is a validated index used for assessing the cutaneous lesions of lupus and consists of 2 separate scores: the first summarizes the inflammatory activity of the disease; the second is a measure of the damage done by the disease. The activity score takes into account erythema, scale/hypertrophy, mucous membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are most often involved in cutaneous lupus erythematosus (CLE). The most severe lesion in each area is measured.

Modified CLASI (mCLASI) is defined as the activity portions of CLASI that describe skin erythema, scale/hypertrophy, and inflammation of the scalp. Activity of oral ulcers and alopecia without scalp inflammation are excluded from the mCLASI analysis, as are all measures of damage. Clinically meaningful improvement in rash, as measured using mCLASI, is defined by ≥50% decrease in baseline activity score.

### 5.8.4. SRI (Systemic Lupus Erythematosus Responder Index of ≥4)

A subject achieves SRI(4) if all of the following criteria are met:
Reduction from baseline of >4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI(X) (X=5, 6, 7, or 8) is defined by the proportion of subjects who meet the following criteria:
Reduction from baseline of ≥X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase ≥0.30 points on a 3-point PGA VAS

### 5.8.5. SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window⁹.

SLEDAI-2K-defined resolution of rash is defined as a score of 0 at Week 52 for those with a score ≥2 for rash at baseline.

### 5.9. Type I IFN Gene Signature (IFNGS)

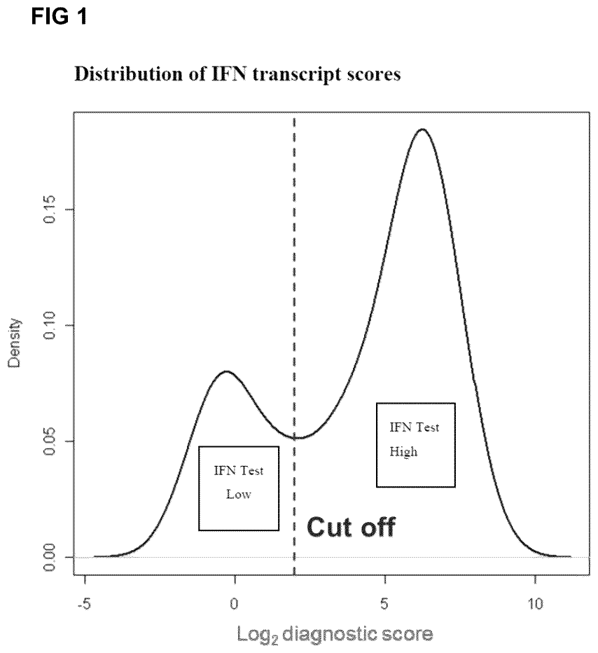

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1). The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (FIG. 1).

The expression of the genes may be measured by RT-PCR. Suitable primers and probes for detection of the genes may be found in WO2011028933. A suitable kit for measuring gene expression for the IFNGS test is the QIAGEN therascreen® IFIGx RGQ RT-PCR kit (IFIGx kit), as described in Brohawn et al.¹⁰, which is incorporated herein by reference in its entirety.

The IFN 21-gene signature (IFNGS) is a validated pharmacodynamic marker of type I IFN signaling [10], that is elevated in patients with type I IFN-mediated disease, including CLE.

A 4-gene IFNGS score is calculated by measurement of IFI27, IFI44, IFI44L, and RSAD2 expression. A 5-gene IFNGS score is calculated by measurement of IFI27, RSAD2, IFI44, IFI44L, IFI6 expression. A 21-gene IFNGS score is calculated by measurement of the genes shown in Error! Reference source not found. Gene expression may be measured by detecting mRNA in the whole blood or tissue of the subject. A IFNGS (4-gene, 5-gene or 21-gene) score may be detected in a subject by measuring the IFNGS gene expression (e.g. mRNA) in the blood or tissue of the subject and comparing the gene expression levels to expression of house-keeping or control genes, e.g. ACTB, GAPDH, and 18S rRNA, in the blood or tissue.

### 6. EXAMPLE 1: MUSE, CLINICALTRIAL.GOV IDENTIFIER: NCT01438489

MUSE was a Phase 2, multinational, multicentre, randomized, double-blind, placebo controlled, parallel-group study to evaluate the efficacy and safety of 2 intravenous (IV) treatment regimens in adult participants with chronic, moderately-to-severely active SLE with an inadequate response to standard of care (SOC) SLE. The investigational product (anifrolumab or placebo) was administered as a fixed dose every 4 weeks (28 days) for a total of 13 doses.

MUSE is described in further detail in Furie et al. 2017³, which is incorporated herein by reference in its entirety.

### 7. EXAMPLE 2: TULIP I AND II, CLINICALTRIAL.GOV IDENTIFIERS: NCT02446912 AND NCT02446899

TULIP I and TULIP II were Phase 3, multicentre, multinational, randomised, double-blind, placebo-controlled studies to evaluate the efficacy and safety of an intravenous (IV) treatment regimen of two doses of anifrolumab versus placebo in subjects with moderately to severely active, autoantibody-positive systemic lupus erythematosus (SLE) while receiving standard of care (SOC) treatment.

### 7.1.1. Restricted Medications

If a subject received 1 of the following, the subject was considered a non-responder. Sulfasalazine; Danazol; Dapsone; Azathioprine >200 mg/day or at a daily dose greater than that at Week 0 (Day 1); Mycophenolate mofetil >2.0 g/day or mycophenolic acid >1.44 g/day or at a daily; dose greater than that at Week 0 (Day 1); Oral, SC, or intramuscular methotrexate >25 mg/week or at a daily dose greater than that at Week 0 (Day 1); Mizoribine >150 mg/day or at a daily dose greater than that at Week 0 (Day 1); Any change in route of administration of oral, SC, or intramuscular methotrexate; Intravenous corticosteroids >40 mg/day but ≤1 gm/day methylprednisolone or equivalent; Intramuscular corticosteroids >80 mg/day methylprednisolone or equivalent; Subcutaneous or intramuscular corticosteroid precursors; Treatment with OCS >40 mg/day prednisone or equivalent; Treatment with OCS above Day 1 dose for a dosing period >14 days; Corticosteroids with a long biologic half-life (eg, dexamethasone, betamethasone); Other immunosuppressants including but not limited to calcineurin inhibitors (eg, cyclosporine, tacrolimus [including topical]) or leflunomide. Cyclosporine eye drops were acceptable for use in the study.

TULIP I is described in further detail in Furie et al. 2019¹¹, which is incorporated herein by reference in its entirety. The results of TULIP II are presented in Morand et al. 2020¹², herein incorporated by reference in its entirety.

### 8. EXAMPLE 3: SKIN FLARE ASSESSMENTS AND OCS TAPER IN LUPUS PATIENTS TREATED WITH ANIFROLUMAB IN 2 PHASE 3 TRIALS

### 8.1. Introduction

Lupus disease flares and lupus treatment with oral corticosteroids (OCS) are associated with organ damage accrual. Patients with lupus who received anifrolumab, a monoclonal antibody to the type I interferon receptor subunit 1, had lower flare rates and were able to taper OCS dosage versus placebo in the phase 3 trials, TULIP-1 (NCT02446912) and TULIP-2 (NCT02446899). The inventors evaluated the effect of anifrolumab treatment on skin flares and in relation to OCS taper in the TULIP trials.

### 8.2. Methods

The randomized, double-blind, placebo-controlled TULIP-1 and TULIP-2 trials evaluated the efficacy and safety of anifrolumab (300 mg IV every 4 weeks for 48 weeks, primary endpoint at Week 52) in patients with moderately to severely active SLE despite standard-of-care treatment. Flares were defined as ≥1 new BILAG-2004 A or ≥2 new BILAG-2004 B domain scores versus the prior visit. An OCS tapering attempt to ≤7.5 mg/day was required between Weeks 8 and 40 for patients receiving baseline OCS ≥10mg/day. Maintained OCS dosage reduction was defined as OCS dosage of ≤7.5 mg/day achieved by Week 40 and maintained to Week 52. TULIP-1 and-2 were analyzed separately using restricted medication rules per the TULIP-2 protocol, and data from both trials were pooled. The inventors analyzed flares descriptively by organ domain and in patients on OCS ≥10 mg/day at baseline with maintained OCS reduction.

### 8.3. Results

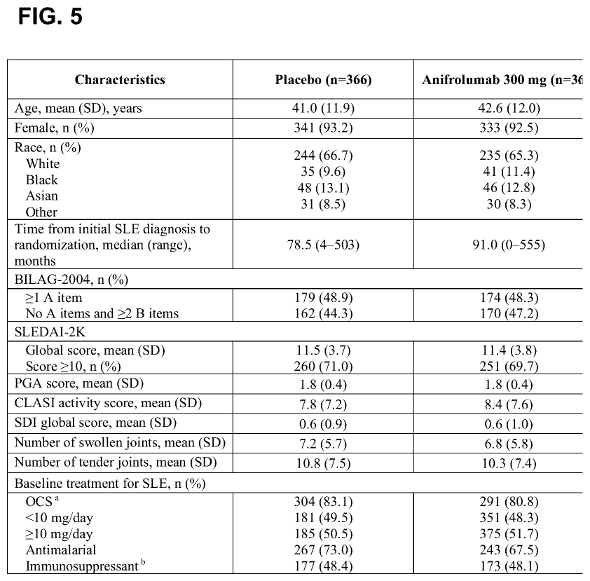

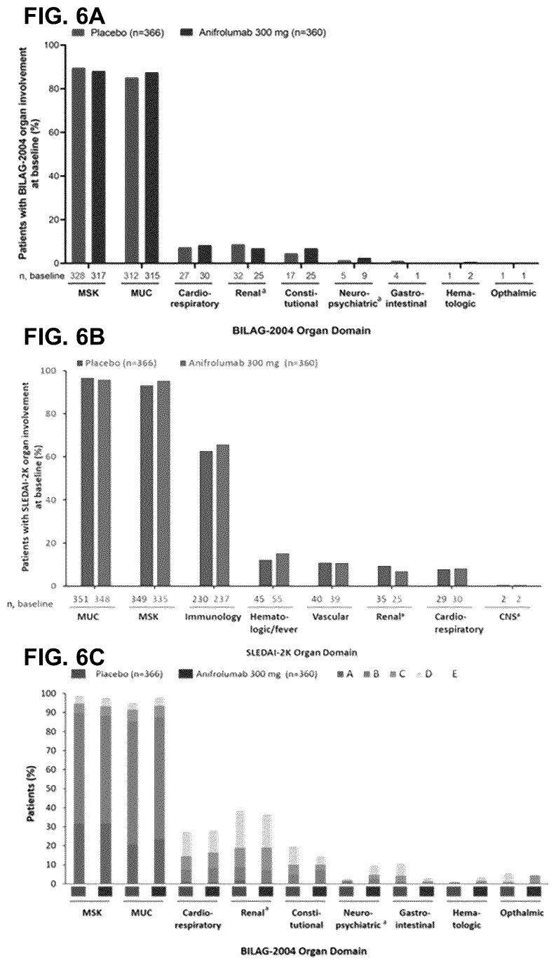

Data were pooled for 726 patients; 360 received anifrolumab 300 mg (180 patients in each trial) and 366 received placebo (184 and 182 patients in TULIP-1 and TULIP-2, respectively). Baseline patient demographics and treatment characteristics were comparable between treatment groups (FIG. 5). In the anifrolumab and placebo arms, the majority of patients had ≥1 BILAG A organ domain score (48.3% and 48.9%, respectively) or no A items and ≥2 B items (47.2% and 44.3%) (FIG. 5). The mean (SD) SLEDAI-2K score was 11.4 (3.8) and 11.5 (3.7) in the anifrolumab and placebo groups, respectively. The most commonly affected organ domains at baseline were mucocutaneous (BILAG-2004 86.4%, n=627; SLEDAI-2K 96.3%, n=699) musculoskeletal (BILAG-2004 88.8%, n=645; SLEDAI-2K 94.2%, n=684), and immunologic (SLEDAI-2K 64.3%, n=467) (FIG. 6A-C); central nervous system (CNS)/neuropsychiatric and renal involvement were relatively uncommon at baseline for both BILAG-2004 (<3%, neuropsychiatric; <8%, renal) and SLEDAI-2K (<0.6%, CNS; <10%, renal) because of the exclusion of patients with severe active lupus nephritis or severe active CNS manifestations. Baseline organ domain involvement assessed by BILAG-2004 and SLEDAI-2K was similar between treatment groups (FIG. 6A-C).

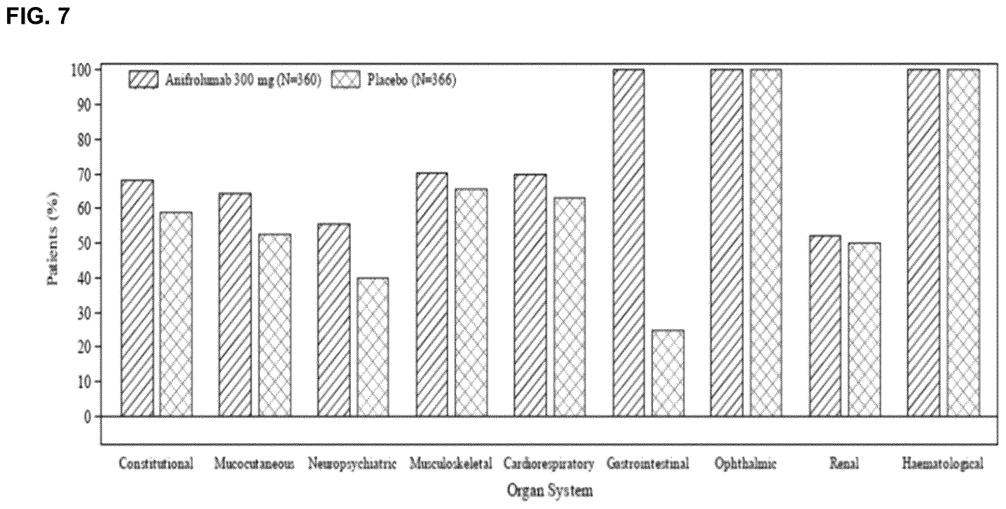

At Week 52, a greater number of patients treated with anifrolumab vs placebo had improvements in the BILAG-2004 mucocutaneous and musculoskeletal domain scores. Improvements were also observed in the majority of less frequently affected domains (FIG. 7).

In total, 360 patients received anifrolumab (TULIP-1, n=180; TULIP-2, n=180) and 366 received placebo (TULIP-1, n=184; TULIP-2, n=182). Overall, fewer patients had ≥1 flare with anifrolumab (33.6%, n=121) versus placebo (42.9%, n=157). Flares occurred most frequently in the mucocutaneous, musculoskeletal, and renal domains in both treatment groups; across all 3 domains, fewer patients experienced ≥1 flare with anifrolumab (22.8%, 19.4%, and 5.0%) versus placebo (26.8%, 25.4%, and 7.4%) (FIG. 7 and Table 8-1).

TABLE 8-1
Number of Flares by Organ Domain in Pooled TULIP-1 and TULIP-2 Data.

| | Overall, n (%) | | | | Mucocutaneous, n (%) | | | | Musculoskeletal, n (%) | | | | Renal, n (%) | | | |
|---|---|---|---|---|---|---|---|---|---|---|---|---|---|---|---|---|
| | | | Anifrolumab | | | | Anifrolumab | | | | Anifrolumab | | | | Anifrolumab | |
| Number | Placebo | | 300 mg | | Placebo | | 300 mg | | Placebo | | 300 mg | | Placebo | | 300 mg | |
| of flares | (n = 366) | | (n = 360) | | (n = 366) | | (n = 360) | | (n = 366) | | (n = 360) | | (n = 366) | | (n = 360) | |
| 0 | 209 | (57.1) | 239 | (66.4) | 268 | (73.2) | 278 | (77.2) | 273 | (74.6) | 290 | (80.6) | 339 | (92.6) | 342 | (95.0) |
| ≥1 | 157 | (42.9) | 121 | (33.6) | 98 | (26.8) | 82 | (22.8) | 93 | (25.4) | 70 | (19.4) | 27 | (7.4) | 18 | (5.0) |
| 1 | 89 | (24.3) | 74 | (20.6) | 68 | (18.6) | 62 | (17.2) | 67 | (18.3) | 48 | (13.3) | 17 | (4.6) | 10 | (2.8) |
| 2 | 49 | (13.4) | 28 | (7.8) | 26 | (7.1) | 18 | (5.0) | 21 | (5.7) | 14 | (3.9) | 7 | (1.9) | 5 | (1.4) |
| ≥3 | 19 | (5.2) | 19 | (5.3) | 4 | (1.1) | 2 | (0.6) | 5 | (1.4) | 8 | (2.2) | 3 | (0.8) | 3 | (0.8) |

BILAG, British Isles Lupus Assessment Group. A flare in the overall group is defined as ≥1 new BILAG-2004 A or ≥2 new BILAG-2004 B items compared with the prior visit. A flare in a BILAG organ domain is present if the respective organ is associated with a flare. Data are presented for organ domains associated with ≥1 flare in ≥5% of patients in the anifrolumab group.

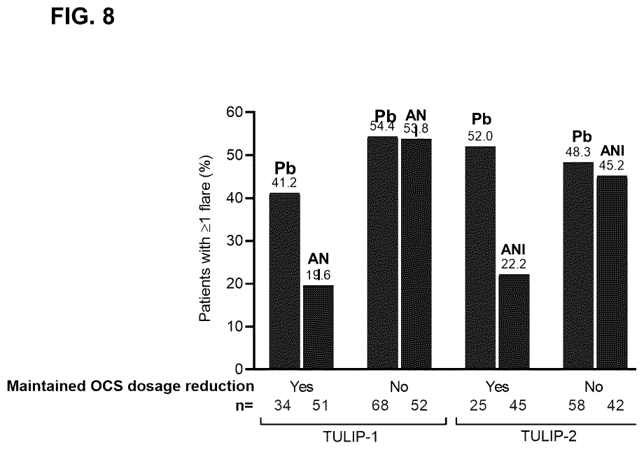

Fewer patients with maintained OCS reduction experienced ≥1 flare with anifrolumab (TULIP-1: 19.6%; TULIP-2: 22.2%) versus placebo (TULIP-1: 41.2%; TULIP-2: 52.0%) (FIG. 8). Similar percentages of patients without maintained OCS reduction experienced ≥1 flare with anifrolumab (TULIP-1: 53.8%; TULIP-2: 45.2%) versus placebo (TULIP-1: 54.4%; TULIP-2: 48.3%).

### 8.4. Conclusions

In the phase 3 TULIP-1 and TULIP-2 trials, fewer patients experienced flares across the 3 most frequently affected organ domains (mucocutaneous, musculoskeletal, and renal) with anifrolumab versus placebo. Anifrolumab was surprisingly associated with a >2-fold reduction in flares in patients with maintained OCS dosage reduction versus placebo. TULIP data support the capacity of anifrolumab to reduce SLE skin (mucocutaneous) flares during OCS taper, an important attribute for the long-term management of patients with SLE. Results of the TULIP-1 and TULIP-2 trials previously demonstrated that patients treated with anifrolumab had higher BICLA responder rates. Both BILAG and SLEDAI are incorporated into the BICLA index. However, BILAG was used for evaluating improving and worsening, and SLEDAI-2K was only used for worsening. Evaluation of individual organ domains as assessed by BILAG-2004and SLEDAI-2K demonstrated that anifrolumab treatment, compared with placebo, was associated with improvement in the mucocutaneous organ domains (skin).

### 9. EXAMPLE 4: ANIFROLUMAB EFFECTS ON RASH FOR PATIENTS WITH SLE, AND IMPACT OF INTERFERON SIGNAL IN POOLED DATA FROM PHASE 3 TRIALS

### 9.1. Background

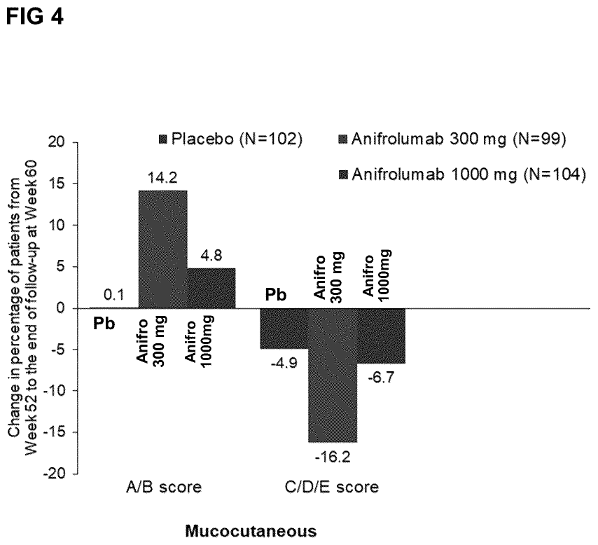

Treatment with anifrolumab is associated with clinical improvements in mucocutaneous disease activity versus placebo in patients with SLE in the phase 2 MUSE trial (NCT01438489) and the phase 3 TULIP trials (FIG. 4 and FIG. 7). The inventors examined symptom-targeted effects of biomarker-defined subsets.

### 9.2. Aim

To evaluate the effect of anifrolumab on rash, and the impact of IFN gene signature (IFNGS) status in patients with SLE using disease measures of different sensitivity in pooled data from the phase 3 TULIP trials.

### 9.3. Methods

TULIP-1 (NCT02446912) and TULIP-2 (NCT02446899) were placebo-controlled, 52-week trials of intravenous anifrolumab administered every 4 weeks in patients with moderate to severe SLE (see Sections 6 and 7). In this post hoc analysis, outcomes of rash and arthritis were evaluated using the mucocutaneous domain of the Systemic Lupus Erythematosus Disease Activity Index 2000 (SLEDAI-2K) (stringent measure) and the British Isles Lupus Assessment Group (BILAG) index (more sensitive measure capturing partial improvements). Improvements in rash, using the modified Cutaneous Lupus Erythematosus Disease Area and Severity Index (mCLASI) score was also evaluated.

### 9.4. Results

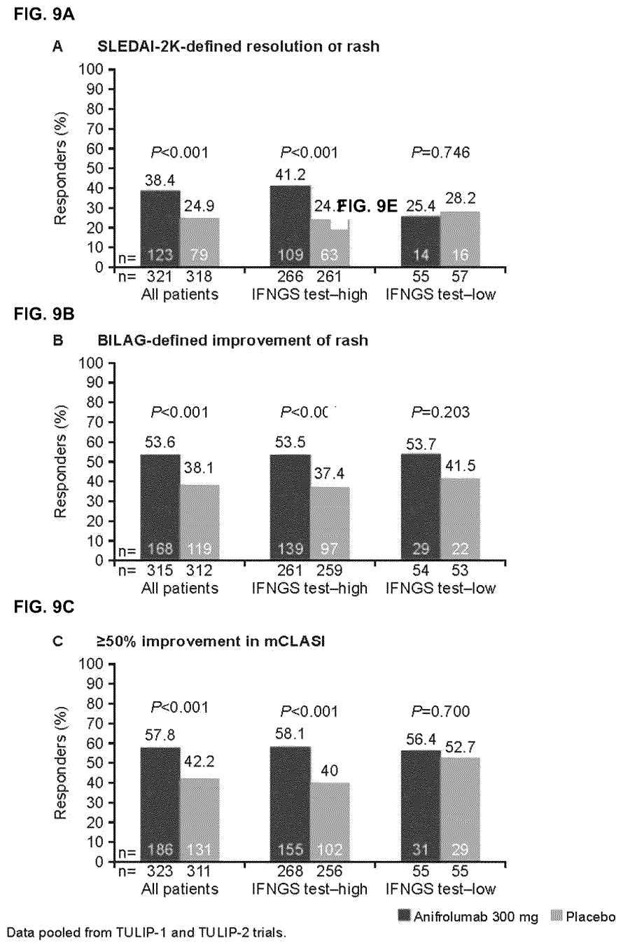

In pooled data from TULIP-1 and TULIP-2, 600 patients (anifrolumab 300 mg, n=298; placebo, n=302) were classified as IFNGS test-high and 126 patients (anifrolumab, n=62; placebo, n=64) as IFNGS test-low. Overall, more anifrolumab-treated patients versus placebo achieved SLEDAI-2K-defined complete resolution of rash (difference 13.5%, nominal P<0.001) (FIG. 9A). The more sensitive measure, BILAG, which required an improvement of ≥1 grade, showed a benefit of anifrolumab over placebo for rash (difference 15.5%, nominal P<0.001); results were comparable in the IFNGS test-high subset (SLEDAI-2K: difference 17%, nominal P<0.001; BILAG: difference 16.1%, nominal P<0.001) (FIG. 9B). In IFNGS test-low patients, there was a trend towards anifrolumab-associated rash improvement. Improvements of ≥50% from baseline to Week 52, defined by mCLASI, in patients with baseline mCLASI activity scores >0, were more frequent with anifrolumab versus placebo (difference 15.6%, nominal P<0.001) (FIG. 9C).

### 9.5. Conclusions

In pooled data from TULIP-1 and TULIP-2, anifrolumab treatment was associated with improvements versus placebo in rash using measures of different stringency.

### 10. EXAMPLE 5: EFFICACY OF ANIFROLUMAB ACROSS ORGAN DOMAINS IN PATIENTS WITH MODERATE TO SEVERE SLE IN POOLED DATA FROM THE TULIP-1 AND TULIP-2 TRIALS

### 10.1. Introduction

The similarity in design of the TULIP-1 and TULIP-2 trials facilitated pooling of data for assessment of individual organ systems with greater statistical power than possible with individual trials alone. In this post hoc analysis of pooled data from the TULIP-1 and TULIP-2 trials, the inventors assessed the effects of anifrolumab on individual SLE organ domain disease activity, particular on the skin.

### 10.2. Methods

### 10.2.1. Patients and Study Design

This was a post hoc analysis of pooled data from the 52-week TULIP-1 and TULIP-2 trials, in which patients who had moderate to severe SLE despite standard therapy with oral glucocorticoids, antimalarials, and/or immunosuppressants were randomized to receive anifrolumab 300 mg or placebo intravenously every 4 weeks for 48 weeks.

The study design and methods have been described in detail previously^{11,12}. In brief, all patients were aged 18 to 70 years and fulfilled the American College of Rheumatology classification criteria for SLE. Patients with active severe neuropsychiatric SLE or severe lupus nephritis were excluded. Mandatory attempts to taper oral glucocorticoids to ≤7.5 mg/day between Week 8 and Week 40 were required for patients receiving prednisone or equivalent ≥10 mg/day at baseline; tapering was also permitted for patients receiving lower doses at baseline. In all patients, glucocorticoid doses were required to be stable from Week 40 through Week 52.

### 10.2.2. Study Endpoints and Assessments

Organ domain involvement was assessed using BILAG-200417 and SLEDAI-2K.18 BILAG-2004response was defined as a reduction from A (severe disease) at baseline to B (moderate), C (mild), or D (no current disease), or from B at baseline to C or D. The proportions of patients who improved 1 step (eg, from A to B or B to C), 2 steps (eg, from A to C or B to D), and up to 3 steps (ie, from A to D) in a given organ domain from baseline to Week 52 were evaluated. SLEDAI-2K improvement was defined as a reduction in domain scores in patients with baseline scores >0. For both BILAG-2004 and SLEDAI-2K, patients who were treated with restricted medication beyond protocol-allowed thresholds or who discontinued investigational product were classified as nonresponders.

Skin and joint disease were further assessed using the Cutaneous Lupus Erythematosus Disease Area and Severity Index (CLASI) activity score (CLASI-A)⁷ and swollen and 28 swollen and tender joint counts, respectively. CLASI response was defined as ≥50% reduction in CLASI-A among patients with baseline CLASI-A ≥10.

In addition to changes in mean hematologic and serologic values, the percentages of patients with abnormal (low or high) values at baseline who converted to normal values at Week 52 were evaluated. Patients who discontinued study treatments or had missing Week 52 data were assumed not to have normalized.

Modified CLASI (mCLASI) is defined as the activity portions of CLASI that describe skin erythema, scale/hypertrophy, and inflammation of the scalp. Activity of oral ulcers and alopecia without scalp inflammation were excluded from the mCLASI analysis, as were all measures of damage. Clinically meaningful improvement in rash, as measured using mCLASI, is defined by ≥50% decrease in baseline activity score.

### 10.2.3. Statistical Analyses

The similar TULIP-1 and TULIP-2 trial designs allowed for the results to be pooled. BILAG-2004 and SLEDAI-2K organ domain responder rates, SLEDAI-2K organ domain responders over time, CLASI-A responders over time, and ≥50% reductions in joint counts from baseline were calculated using a stratified Cochran-Mantel-Haenszel approach, with stratification factors (matching those in the TULIP studies) of SLEDAI-2K score at screening, type I IFN gene signature test status at screening, and Day 1 oral glucocorticoid dose. The reported 2-sided P-values and 95% confidence intervals (CIs) are based on this approach. All reported P-values are nominal. For assessment of pooled TULIP data, TULIP-1 data were analysed according to the TULIP-2-revised restricted medication analytic rules. Missing data were imputed using the last observation carried forward for the first visit with missing data; subsequent visits with missing data were not imputed.

### 10.3. Results

### 10.3.1. Patient Demographics and SLE Medications at Baseline

Data were pooled for 726 patients; 360 received anifrolumab 300 mg IV Q4W (180 patients in each trial) and 366 received placebo (184 patients in TULIP-1 and 182 patients in TULIP-2). In the pooled population, 82.6% (600/726) of patients were found to be IFNGS high and 17.4% (126/726) were IFNGS low (Table 1). Baseline patient demographics were comparable between treatment groups (Table 10-1). Of the 726 patients enrolled, the mean age was 41.8 years and 92.8% were female. For IFNGS-high and IFNGS-low patients, the median age was 41.1 and 45.1 years and 92.5% and 94.4% were female.

SLE-related treatments were similar between treatment groups (Table 10-1). At baseline, the majority of patients (82.0%) were receiving glucocorticoids and more than 50% in both treatment groups were receiving ≥10 mg/day. Similar proportions of patients receiving anifrolumab or placebo were also receiving immunosuppressants (48.1% vs 48.4%,) and antimalarial treatment (67.5% vs 73.0%). More IFNGS-high patients than IFNGS-low patients were taking glucocorticoids of any dose (84.7% vs 69.0%) and ≥10 mg/day (54.7% vs 37.3%). This was also the case for immunosuppressants (51.0% vs 34.9%), but fewer IFNGS-high than IFNGS-low patients received antimalarials (68.2% vs 80.2%).

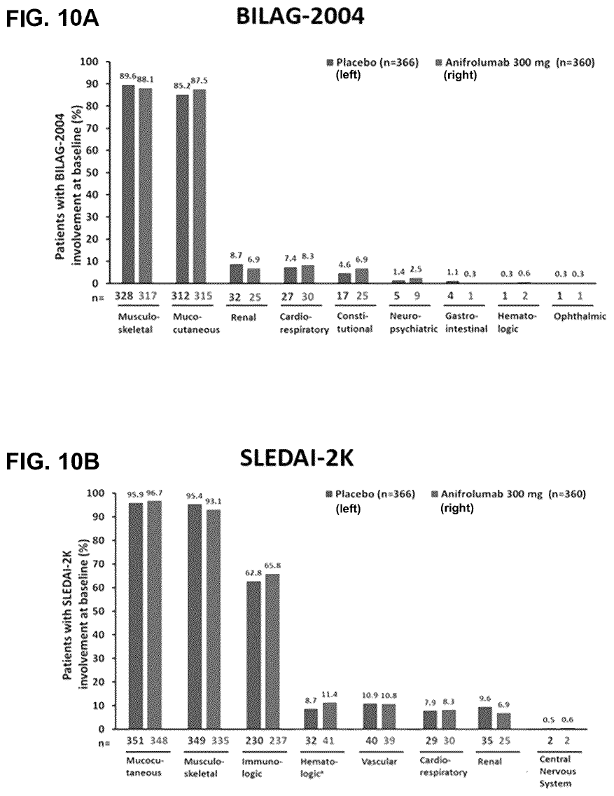

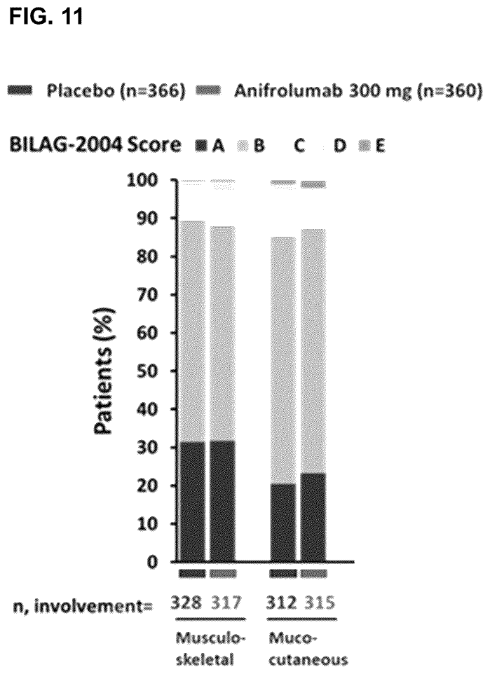

Baseline organ domain involvement assessed using BILAG-2004 and SLEDAI-2K was similar between treatment groups (FIG. 10A and FIG. 10B). The most commonly affected organ domains at baseline were mucocutaneous (BILAG-2004 86.4% [627/726]; SLEDAI-2K 96.3% [699/726]), musculoskeletal (BILAG-2004 88.8% [645/726]; SLEDAI-2K 94.2% [684/726]), and immunologic (SLEDAI-2K 64.3% [467/726]) (FIG. 10A and FIG. 10B); immunologic variables are not captured in BILAG-2004. Renal and neuropsychiatric involvement were relatively uncommon at baseline regardless of whether the assessments were conducted with BILAG-2004 or SLEDAI-2K. In the most commonly affected BILAG-2004 domains, musculoskeletal and mucocutaneous, the majority of patients had severe or moderate disease activity at baseline as shown by the overall frequency of BILAG A (musculoskeletal 31.5% [229/726], mucocutaneous 21.9% [159/726]) or BILAG B (musculoskeletal 57.3% [416/726]; mucocutaneous 64.5% [468/726]) scores. BILAG organ domain scores were balanced across treatment groups (FIG. 11); SLEDAI-2K cannot discern the severity of activity within an organ domain.

### 10.3.2. Patient Disease Characteristics: Rash

In the anifrolumab and placebo groups, together with the IFNGS-high and-low subgroups, ≥85% of patients had rash at baseline, measured using SLEDAI-2K or BILAG-2004 (Table 10-1). Mean (SD) mCLASI activity scores at baseline were 6.2 (7.03) in the anifrolumab group and 5.8 (6.88) in the placebo group. IFNGS-high patients had greater mCLASI scores than did IFNGS-low patients at baseline (mean [SD]: 6.6 [7.50] vs 4.2 [3.5] in the anifrolumab group and 5.9 [7.06] vs 5.3 [4.79] in the placebo group). In the anifrolumab and placebo groups, together with the IFNGS-high and -low subgroups, activity scores >0 were observed in ≥85% of patients. Twenty percent of patients in both the anifrolumab and placebo groups had an activity score ≥10 (Table 10-1). In the IFNGS-high versus IFNGS-low patient subsets, respectively, 22.8% versus 9.7% in the anifrolumab group and 20.9% versus 17.2% in the placebo group had an activity score of ≥10.

TABLE 10-1
Baseline Characteristics for Clinical Outcome Measures

| | Placebo | | | | | | Anifrolumab 300 mg | | | | | |
|---|---|---|---|---|---|---|---|---|---|---|---|---|
| | Total | | IFNGS | | IFNGS | | Total | | IFNGS | | IFNGS | |
| | population | | high | | low | | population | | high | | low | |
| Characteristics | (n = 366) | | (n = 302) | | (n = 64) | | (n = 360) | | (n = 298) | | (n = 62) | |
| Age, mean (SD), years | 41.0 | (11.9) | 40.7 | (11.7) | 42.7 | (12.6) | 42.6 | (12.0) | 41.5 | (11.9) | 47.6 | (11.3) |
| Female, n (%) | 341 | (93.2) | 280 | (92.7) | 61 | (95.3) | 333 | (92.5) | 275 | (92.3) | 58 | (93.5) |
| Race, n (%) | | | | | | | | | | | | |
| White | 244 | (66.7) | 192 | (63.6) | 52 | (81.3) | 235 | (65.3) | 184 | (61.7) | 51 | (82.3) |
| Black | 48 | (13.1) | 40 | (13.2) | 8 | (12.5) | 46 | (12.8) | 41 | (13.8) | 5 | (8.1) |
| Asian | 35 | (9.6) | 33 | (10.9) | 2 | (3.1) | 41 | (11.4) | 39 | (13.1) | 2 | (3.2) |
| Other | 31 | (8.5) | 29 | (9.6) | 2 | (3.1) | 30 | (8.3) | 27 | (9.1) | 3 | (4.8) |
| Time from initial | 78.5 | (4-503) | 87.5 | (4-503) | 52.0 | (6-389) | 91.0 | (0-555) | 97.0 | (6-555) | 77.0 | (0-388) |
| SLE diagnosis to | | | | | | | | | | | | |
| randomization, median | | | | | | | | | | | | |
| (range), months | | | | | | | | | | | | |

Baseline treatment for SLE, n (%)

| | | | | | | | | | | | | |
|---|---|---|---|---|---|---|---|---|---|---|---|---|
| Oral glucocorticoids^{a} | 304 | (83.1) | 257 | (85.1) | 47 | (73.4) | 291 | (80.8) | 251 | (84.2) | 40 | (64.5) |
| <10 mg/day | 181 | (49.5) | 97 | (32.1) | 22 | (34.4) | 351 | (48.3) | 83 | (27.9) | 18 | (29.0) |
| ≥10 mg/day | 185 | (50.5) | 160 | (53.0) | 25 | (39.1) | 375 | (51.7) | 168 | (56.4) | 22 | (35.5) |
| Antimalarial | 267 | (73.0) | 214 | (70.9) | 53 | (82.8) | 243 | (67.5) | 185 | (65.4) | 48 | (77.4) |
| Immunosuppressant^{b} | 177 | (48.4) | 157 | (52.0) | 20 | (31.3) | 173 | (48.1) | 149 | (50.0) | 24 | (31.3) |

Rash measured using SLEDAI-2K, n (%)

| | | | | | | | | | | | | |
|---|---|---|---|---|---|---|---|---|---|---|---|---|
| Present | 318 | (86.9) | 261 | (86.4) | 57 | (89.1) | 321 | (89.2) | 266 | (89.3) | 55 | (88.7) |

Rash measured using BILAG-2004, n (%)

| | | | | | | | | | | | | |
|---|---|---|---|---|---|---|---|---|---|---|---|---|
| A | 75 | (20.5) | 63 | (20.9) | 12 | (18.8) | 84 | (23.3) | 71 | (23.8) | 13 | (21.0) |
| B | 237 | (64.8) | 196 | (64.9) | 41 | (64.1) | 231 | (64.2) | 190 | (63.8) | 41 | (66.1) |
| C | 44 | (12.0) | 34 | (11.3) | 10 | (15.6) | 34 | (9.4) | 28 | (9.4) | 6 | (9.7) |
| D/E | 10 | (2.7) | 9 | (3.0) | 1 | (1.6) | 11 | (3.1) | 9 | (3.0) | 2 | (3.2) |

Rash measured using mCLASI, n (%)

| | | | | | | | | | | | | |
|---|---|---|---|---|---|---|---|---|---|---|---|---|
| Activity score >0 | 311 | (85.0) | 256 | (84.8) | 55 | (85.9) | 323 | (89.7) | 268 | (89.9) | 55 | (88.7) |
| Activity score ≥10 | 74 | (20.2) | 63 | (20.9) | 11 | (17.2) | 74 | (20.6) | 68 | (22.8) | 6 | (9.7) |

BILAG-2004, British Isles Lupus Assessment Group-2004; mCLASI, modified Cutaneous Lupus Erythematosus Disease Area and Severity Index; IFNGS, interferon gene signature; Q4W, every 4 weeks; SD, standard deviation; SLEDAI-2K, Systemic Lupus Erythematosus Disease Activity Index 2000.
Rash is considered present when measured using SLEDAI-2K if the rash component of SLEDAI-2K is ≥2. Arthritis is considered present when measured by SLEDAI-2K if the arthritis component of SLEDAI-2K is ≥4. A, severe disease; B, moderate disease; C, mild disease; D/E, clear of disease activity.
^{a}Oral glucocorticoids contains prednisone or equivalent.
^{b}Immunosuppressant: azathioprine, methotrexate, mycophenolate mofetil, mycophenolic acid, and mizoribine.

### 10.3.3. Efficacy in BILAG-2004 Organ Domains

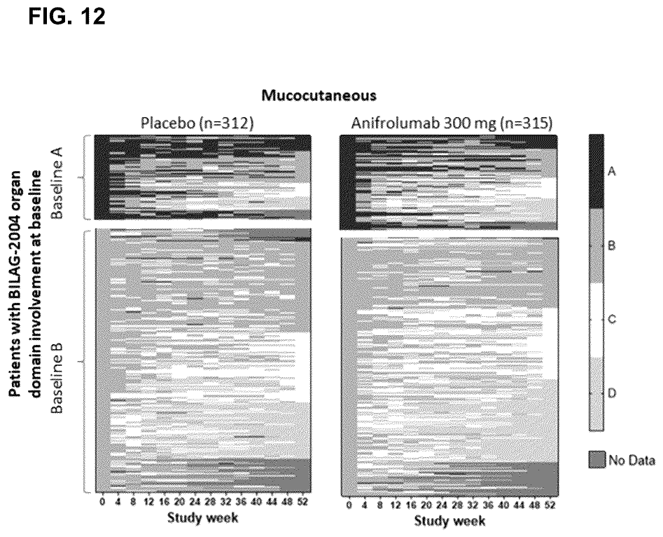

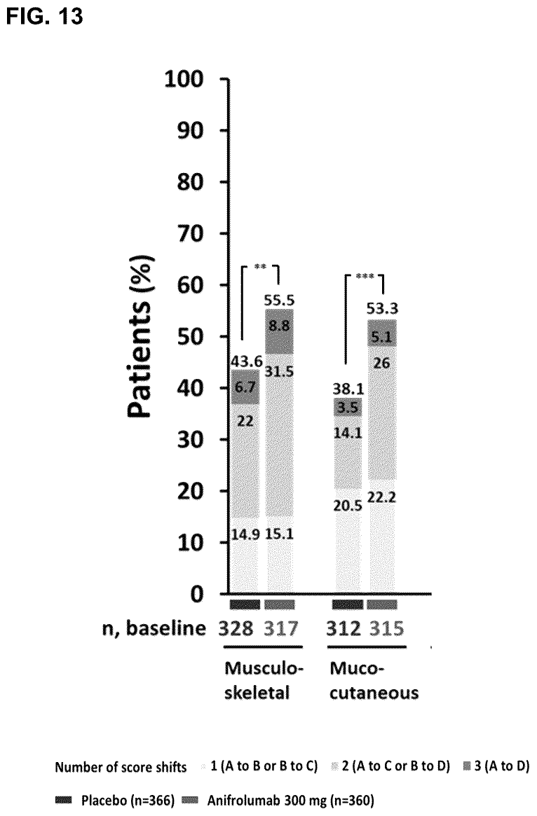

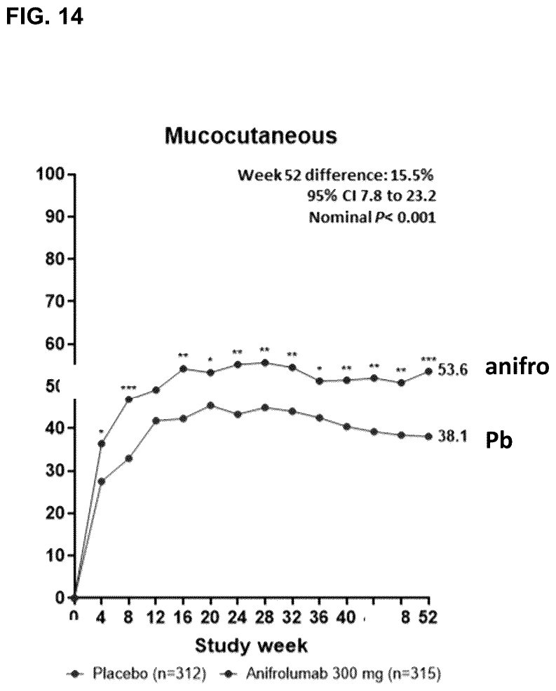

BILAG-2004 patient-level organ domain scores obtained every 4 weeks across the entire trial period are displayed using heat maps (FIG. 12). At Week 52, 55.5% (176/317) of anifrolumab-treated patients achieved a BILAG-2004 musculoskeletal response compared with 43.6% (143/328) of patients receiving placebo (difference 11.9%; 95% CI 4.2, 19.4; nominal P<0.01) (FIGS. 13), and 53.3% (168/315) of patients treated with anifrolumab versus 38.1% (119/312) of patients receiving placebo achieved a BILAG-2004 mucocutaneous response (difference 15.5%; 95% CI 7.8, 23.2; nominal P<0.001) (FIG. 13). Improvements favoring anifrolumab for the mucocutaneous BILAG-2004 domain was observed from Week 4 and Week 32, respectively (both P<0.05) (FIG. 14).

### 10.3.4. Efficacy in SLEDAI-2K Organ Domains

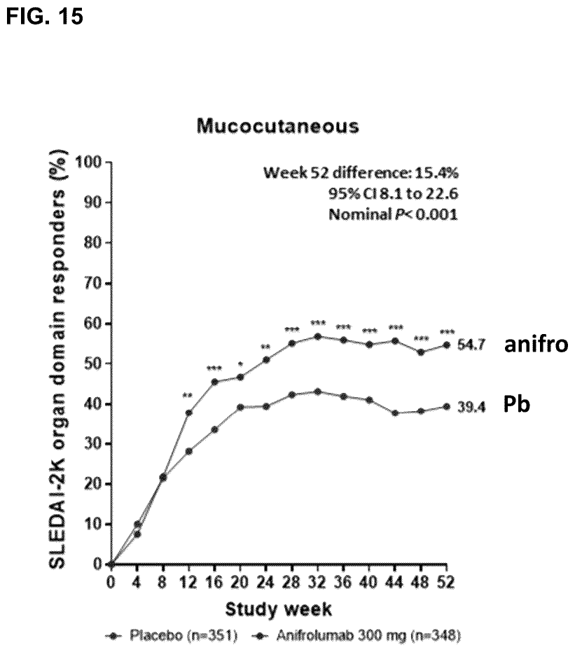

At Week 52, significantly more anifrolumab-treated than placebo-receiving patients had improvements in the SLEDAI-2K organ domains most frequently affected at baseline: mucocutaneous (54.7% [190/348] vs 39.4% [138/351]; nominal P<0.001), musculoskeletal (48.8% [164/335] vs 40.4% [141/349]; nominal P<0.05), and immunologic (18.6% [44/237] vs 11.3% [26/230]; nominal P<0.05) (FIG. 15). Improvements favoring anifrolumab for the mucocutaneous SLEDAI-2K domain was observed from Week 12 (P<0.05) (FIG. 15).

### 10.3.5. Efficacy of Anifrolumab for Improvement of Rash

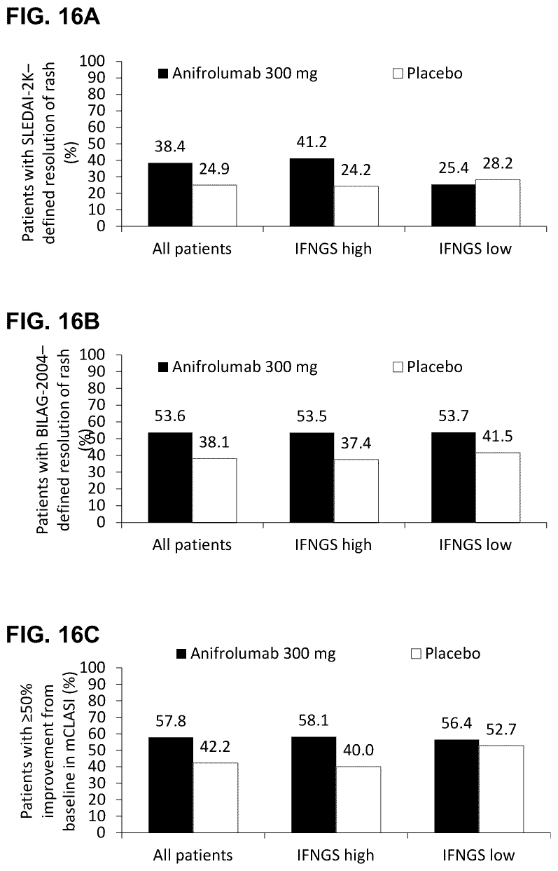

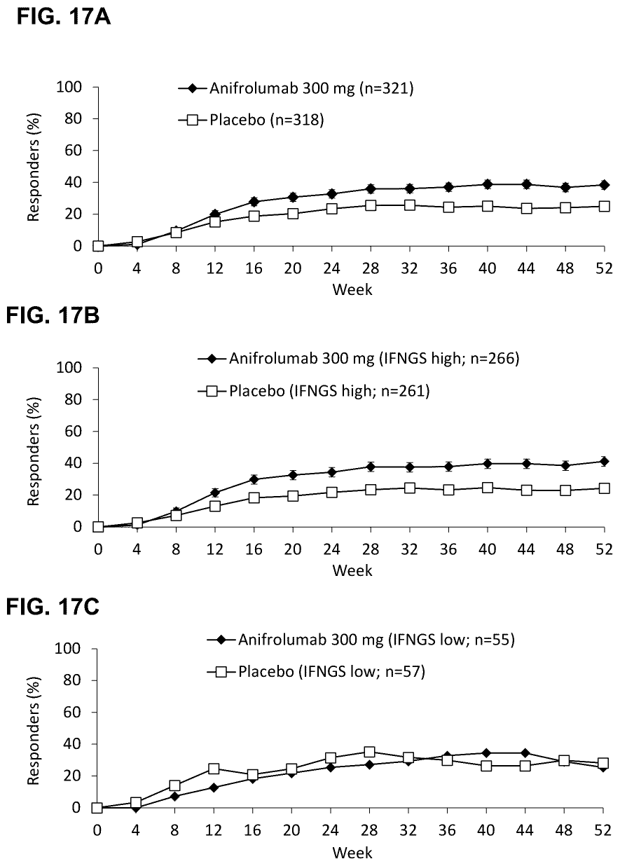

Resolution of rash defined by SLEDAI-2K was achieved by more anifrolumab-treated patients than those receiving placebo at Week 52 (difference 13.5%; nominal P<0.001) (Table 10-2, FIG. 16). These data were driven by the IFNGS-high subset (difference 17.0%; nominal P<0.001) but not the IFNGS-low subset (difference—2.8%; nominal P=0.746) (Table 10-2, FIG. 16A). SLEDAI-2K-defined resolution of rash was more frequent with anifrolumab versus placebo as early as Week 12, and this difference was sustained through to Week 52 (FIG. 17A). Results were comparable in the IFNGS-high subset (FIG. 17B), but there was less separation between treatment groups and higher placebo response among IFNGS-low patients throughout the study (FIG. 17C).

Improvement of rash, assessed using BILAG (≥1 severity grade lowering) was reached by more anifrolumab-treated patients than placebo-treated patients at Week 52 (Table 10-2, FIG. 16B). Results were again similar in the IFNGS-high subset (difference 16.1%; nominal P<0.001), and there was a trend toward anifrolumab-associated rash improvement in IFNGS-low patients (difference 12.2%; nominal P=0.203) (Table 10-2, FIG. 16B). BILAG-2004-defined improvement of rash was greater with anifrolumab than placebo at all time points, as early as Week 4 (FIG. 17A). This was observed similarly in the IFNGS-high subset (FIG. 17B), but, in IFNGS-low patients, the separation between treatment groups was less apparent, and the placebo response was higher than that among IFNGS-high patients (FIG. 17AC).

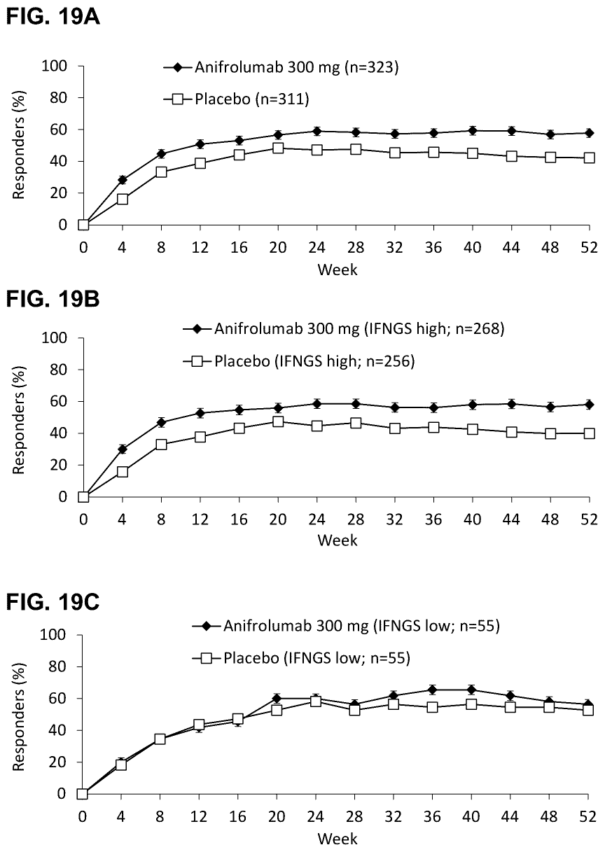

As assessed using mCLASI, more anifrolumab-treated patients with baseline score >0 achieved ≥50% improvement of rash at Week 52 (difference 15.6%; nominal P<0.001) (Table 10-2, FIG. 16C). Results were comparable in IFNGS-high patients (difference 18.1%; nominal P<0.001) but not in IFNGS-low patients (difference 3.6%; nominal P=0.700) (Table 10-2, FIG. 16C). In smaller subsets of patients with baseline mCLASI scores ≥6 or ≥10, there were similar trends (Table 10-2). mCLASI improvement in rash (in patients with mCLASI activity scores >0) was seen as early as Week 4 (FIG. 19A). Improvements were greater with anifrolumab than placebo at all time points, which was also observed for IFNGS-high patients (FIG. 19B) but was not as evident for IFNGS-low patients owing to the high placebo responses (FIG. 19C).

TABLE 10-2
Clinical Outcome Measures of Rash at Week 52

| | | | Anifrolumab 300 mg | | | Placebo | | |
|---|---|---|---|---|---|---|---|---|
| | | | | IFNGS | IFNGS | | IFNGS | IFNGS |
| | | | Total | high | low | Total | high | low |
| SLEDAI-2K | | n | 321 | 266 | 55 | 318 | 216 | 57 |
| | | Responders, n | 123 (38.4) | 109 (41.2) | 14 (25.4) | 79 (24.9) | 63 (24.2) | 16 (28.2) |
| | | (%) [SE] | [2.71] | [3.02] | [6.07] | [2.48] | [2.71] | [6.10] |
| | | Difference [SE] | 13.5 | 17.0 | −2.8 | — | — | — |
| | | (95% CI) | [3.67] | [4.05] | [8.61] | | | |
| | | | (6.3 to 20.7) | (9.1 to 25.0) | (−19.7 to 14.1) | | | |
| | | P-value | <0.001 | <0.001 | 0.746 | — | — | — |
| BILAG-2004 | | n | 315 | 261 | 54 | 312 | 259 | 53 |
| | | Responders, n | 168 (53.6) | 139 (53.5) | 29 (53.7) | 119 (38.1) | 97 (37.4) | 22 (41.5) |
| | | (%) [SE] | [2.79] | [3.06] | [6.78] | [2.75] | [3.01] | [6.79] |
| | | Difference [SE] | 15.5 | 16.1 | 12.2 | — | — | — |
| | | (95% CI) | [3.92] | [4.29] | [9.59] | | | |
| | | | (7.8 to 23.2) | (7.7 to 24.6) | (6.6 to 31.0) | | | |
| | | P-value | <0.001 | <0.001 | 0.203 | — | — | — |
| mCLASI | >0 at | n | 323 | 268 | 55 | 311 | 256 | 55 |
| | baseline | Responders, n | 186 (57.8) | 155 (58.1) | 31 (56.4) | 131 (42.2) | 102 (40) | 29 (52.7) |
| | | (%) [SE] | [2.74] | [3.00] | [6.70] | [2.78] | [3.07] | [6.64] |
| | | Difference [SE] | 15.6 | 18.1 | 3.6 | — | — | — |
| | | (95% CI) | [3.91] | [4.29] | [9.43] | | | |
| | | | (8.0 to 23.3) | (9.7 to 26.6) | (−14.8 to 22.1) | | | |
| | | P-value | <0.001 | <0.001 | 0.700 | — | — | — |
| | ≥6 at | n | 141 | 121 | 20 | 135 | 113 | 22 |
| | baseline | Responders, n | 86 (61.2) | 73 (60.5) | 13 (65.0) | 65 (47.9) | 50 (44.3) | 15 (68.1) |
| | | (%) [SE] | [4.14] | [4.47] | [10.92] | [4.25] | [4.66] | [10.28] |
| | | Difference [SE] | 13.3 | 16.2 | −3.1 | — | — | — |
| | | (95% CI) | [5.93] | [6.46] | [15.00] | | | |
| | | | (1.6 to 24.9) | (3.6 to 28.9) | (−32.5 to 26.3) | | | |
| | | P-value | 0.025 | 0.012 | 0.835 | — | — | — |
| | ≥10 at | n | 74 | 68 | 6 | 74 | 63 | 11 |
| | baseline | Responders, n | 45 (60.9) | 40 (58.9) | 5 (85.0) | 35 (47.4) | 26 (41.5) | 9 (80.9) |
| | | (%) [SE] | [5.70] | [5.99] | [19.26] | [5.81] | [6.21] | [14.29] |
| | | Difference [SE] | 13.5 | 17.4 | 4.1 | — | — | — |
| | | (95% CI) | [8.14] | [8.63] | [23.99] | | | |
| | | | (−2.4 to 29.5) | (0.5 to 34.3) | (−42.9 to 51.1) | | | |
| | | P-value | 0.097 | 0.043 | 0.864 | — | — | — |

BILAG-2004, British Isles Lupus Assessment Group-2004; mCLASI, modified Cutaneous Lupus Erythematosus Disease Area and Severity Index; IFNGS, interferon gene signature; SE, standard error; SLEDAI-2K, Systemic Lupus Erythematosus Disease Activity Index 2000.

### 10.3.6. Laboratory Markers—Hematology and Serology

Patients in the anifrolumab and placebo groups had similar mean hematology values at baseline (Table 10-3).

TABLE 10-3
Changes in hematologic measures from baseline to Week 52

| | |
|---|---|
| Placebo | Anifrolumab 300 mg |
| (n = 365)^{a} | (n = 360) |

Hemoglobin

| | | |
|---|---|---|
| Baseline mean (SD), g/L | 126.0 (15.2) | 125.0 (14.8) |
| Change from baseline, | −2.7 (11.33) | 0.5 (10.59) |
| mean (SD), g/L | | |
| Normalization at Week 52 | 0 (0) | 0 (0) |
| in patients with abnormal | | |
| hemoglobin at baseline, n (%)^{b} | | |

Hematocrit

| | | |
|---|---|---|
| Baseline mean (SD) | 0.4 (0.04) | 0.4 (0.04) |
| Change from baseline, mean (SD) | −0.005 (0.03) | 0.005 (0.03) |
| Normalization at Week 52 in | 0 (0) | 0 (0) |
| patients with abnormal | | |
| hematocrit at baseline, n (%)^{b} | | |

Lymphocytes

| | | |
|---|---|---|
| Baseline mean (SD), 10⁹/L | 1.3 (0.6) | 1.3 (0.6) |
| Change from baseline, | −0.03 (0.5) | 0.3 (0.6) |
| mean (SD), 10⁹/L | | |
| Normalization at Week 52 in | 11 (3.0) | 23 (6.4) |
| patients with abnormal | | |
| lymphocytes at baseline, n (%)^{b} | | |

Neutrophils

| | | |
|---|---|---|
| Baseline mean (SD), 10⁹/L | 4.0 (2.1) | 3.8 (1.8) |
| Change from baseline, | 0.1 (2.0) | 0.7 (1.8) |
| mean (SD), 10⁹/L | | |
| Normalization at Week 52 | 0 (0) | 1 (0.3) |
| in patients with abnormal | | |
| neutrophils at baseline, n (%)^{b} | | |

Platelets

| | | |
|---|---|---|
| Baseline mean (SD), 10⁹/L | 250.2 (79.8) | 239.9 (78.2) |
| Change from baseline, | 3.2 (49.8) | 24.3 (58.2) |
| mean (SD), 10⁹/L | | |
| Normalization at Week 52 in | 1 (0.3) | 0 (0.0) |
| patients with abnormal | | |
| platelets at baseline, n (%)^{b} | | |

SD, standard deviation.
^{a}1 patient was removed from the analysis after study completion;
^{b}Range of normal values for hemoglobin (>60 to <200 g/L), hematocrit (>0.18 to <0.64), lymphocytes (>0.5 to <10.0 109/L), neutrophils (>0.5 to <20.0 109/L), and platelets (>20 to <600 109/L).

At Week 52, treatment effects favoring anifrolumab versus placebo were seen for mean (SD) increase in haemoglobin (0.5 [10.59] vs −2.7 [11.33] g/L) and platelets (24.3 [58.2] vs 3.2 [49.8] ×109/L). In the anifrolumab group, 6.4% (23/360) of patients with leukopenia at baseline demonstrated normalization, versus 3.0% (11/366) of patients receiving placebo.

Among patients who were anti-dsDNA positive at baseline, mean (SD) levels of anti-dsDNA antibodies decreased with anifrolumab treatment, compared with an increase for placebo (−25.0 [238.4] vs 28.0 [498.5] U/mL). Accordingly, 7.8% (13/167) of patients receiving anifrolumab versus 5.8% (9/155) of patients receiving placebo converted to anti-dsDNA negative by Week 52 (Table 10-4).

At Week 52, greater improvements from baseline in mean (SD) complement C3 levels were observed with anifrolumab (0.13 [0.18]) versus placebo (0.04 [0.16] U/mL) (Table 10-4). In patients with low C3 at baseline, normalization was observed in 16.2% (21/130) of anifrolumab-treated and 9.5% (13/137) of placebo-treated patients. Similarly, normalization of low baseline C4 occurred in more patients receiving anifrolumab versus placebo (22.6% [19/84] vs 7.1% [6/85]).

TABLE 10-4
Change in laboratory markers from baseline to Week 52

| | |
|---|---|
| | Anifrolumab |
| Placebo | 300 mg |
| (n = 366) | (n = 360) |

Anti-dsDNA^{a,b}

| | | |
|---|---|---|
| Anti-dsDNA positive at baseline, n (%) | 155 (42.3) | 167 (43.4) |
| Mean (SD), U/mL | 211.95 (549.65) | 129.34 (261.40) |
| Change from baseline, | 27.96 (498.47) | −24.98 (238.39) |
| mean (SD), U/mL | | |
| Normalization at Week 52 in | 9 (5.8%) | 13 (7.8%) |
| patients with abnormal | | |
| anti-dsDNA at baseline, n (%) | | |

C3^{a,c}

| | | |
|---|---|---|
| Abnormal C3 at baseline, n (%) | 137 (37.4) | 130 (36.1) |
| Mean (SD), U/mL | 0.70 (0.14) | 0.69 (0.15) |
| Change from baseline, | 0.04 (0.16) | 0.13 (0.18) |
| mean (SD), U/ml | | |
| Normalization at Week 52 in | 13 (9.5) | 21 (16.2) |
| patients with abnormal | | |
| C3 at baseline, n (%) | | |

C4^{a,d}

| | | |
|---|---|---|
| Abnormal C4 at baseline, n (%) | 85 (23.2) | 84 (23.3) |
| Mean (SD), U/mL | 0.07 (0.02) | 0.07 (0.02) |
| Change from baseline, | 0.02 (0.04) | 0.02 (0.03) |
| mean (SD), U/ml | | |
| Normalization at Week 52 in | 6 (7.1) | 19 (22.6) |
| patients with abnormal | | |
| C4 at baseline, n (%) | | |

anti-dsDNA, anti-double-stranded DNA;
C3, complement 3;
C4, complement 4;
SD, standard deviation.
^{a}Only patients with baseline positive anti-dsDNA or low C3 or C4 are included in the summary statistics for the respective variables;
^{b}Anti-dsDNA antibody “positive” defined as a result of >15 U/mL;
^{c}Complement C3 “abnormal” levels defined as a result of <0.9 g/L;
^{d}Complement C4 “abnormal” levels defined as a result of <0.1 g/L.

### 10.4. Discussion

In this post hoc analysis of pooled data from the TULIP-1 and TULIP-2 trials, compared with placebo, anifrolumab treatment was associated with greater improvement in the most frequently affected organ domains (mucocutaneous and musculoskeletal) of patients with moderate to severe SLE. Anifrolumab treatment also resulted in greater improvements in skin disease and in greater frequency of hematologic and serologic normalization compared with placebo.

Results of the TULIP-1 and TULIP-2 trials previously demonstrated that patients treated with anifrolumab had higher BICLA responder rates compared with patients receiving placebo. The present analyses also surprisingly demonstrate consistency between BILAG-2004 and SLEDAI-2K activity assessments for skin disease. Baseline involvement of the mucocutaneous domain was present in >85% of patients as determined using BILAG-2004 and >90% using SLEDAI-2K. Using either BILAG-2004 or SLEDAI-2K organ domain responder assessments, greater improvement in the mucocutaneous domain were observed with anifrolumab versus placebo, and improvements within these domains were comparable between indices.

In addition to analysis of BILAG-2004 and SLEDAI-2K mucocutaneous domains, the inventors used a validated skin-specific tool, CLASI, to assess skin disease in patients with SLE. The inventors assessed CLASI response in the subset of patients with CLASI-A ≥10 at baseline to focus on the patients with moderate to severe skin disease. Using all 3 skin disease measures, the inventors observed a robust and early improvement of skin involvement in anifrolumab-treated patients compared with placebo.

Serologic activity is indicative of immune system activation and is typically associated with SLE disease activity. More anifrolumab-treated patients were able to normalize anti-dsDNA antibodies and complement C3 and C4 levels compared with placebo-treated patients. These results suggest that the effects of anifrolumab on serologic markers are consistent with the greater improvements observed in those treated with anifrolumab compared with placebo in the SLEDAI-2K immunologic domain.

The phase 3 TULIP trials found that treatment with anifrolumab 300 mg IV Q4W was associated with improvements in both rash and arthritis compared with placebo for patients with SLE, using measures of different stringency. SLEDAI-2K-defined resolution of rash and arthritis is very stringent and requires complete resolution of these symptoms. Evaluation of rash and arthritis by SLEDAI-2K demonstrated that more patients treated with anifrolumab compared with placebo achieved resolution. BILAG-defined improvement in rash allows for detection of partial improvements and, therefore, is more sensitive to change. Improvements were detected with BILAG for rash as a result of anifrolumab treatment. Evaluation of mCLASI scores also allows for detection of partial improvements in rash. This instrument is quite flexible in its ability to detect improvement with sensitivity or stringency, depending on the degree of rash at entry and the degree of change used as a cutoff for improvement. Assessment of mCLASI scores over time demonstrated that anifrolumab treatment, compared with placebo, was associated with improvement early in treatment. Evaluation of joint counts allows for detection of partial improvements in arthritis and, similarly, is flexible in its ability to detect improvement with sensitivity or stringency, as joint counts at entry and the degree of change selected for improvement can be modified. Tender/swollen joint counts improved as a result of anifrolumab treatment.

Results also suggested that patients who were IFNGS high at baseline responded better to anifrolumab treatment versus placebo than did IFNGS-low patients with regards to improvements in rash and arthritis. Across all disease measures for both rash and arthritis, improvements at Week 52 for IFNGS-high patients were comparable to results from the total population, and the time course of these improvements closely followed those observed in the total population. The majority of patients were IFNGS high at baseline; therefore, it would be expected that the trends seen in the total population would be reflective of this majority subgroup. The high proportion of IFNGS-high patients is unsurprising, as these patients are more likely to enter a randomized trial owing to the difficulty of managing their disease with standard therapy, 10-13 as illustrated by these patients having high baseline glucocorticoid doses. Among IFNGS-low patients, treatment response to anifrolumab for rash and arthritis did appear to increase when measured using more sensitive instruments, suggesting some benefit of treatment for this subgroup.

These analyses particularly support the hypothesis that type I IFN is a key driver for skin disease in SLE. The effect of the IFN signal on mCLASI over time becomes apparent early in treatment as a difference is seen between anifrolumab and placebo among IFNGS-high patients as early as Week 4. This separation is not seen until Week 20 among IFNGS-low patients. However, there was a trend toward some anifrolumab-associated improvements in rash in IFNGS-low patients using more sensitive measures. IFN activity may not always be reflective of levels of IFNGS expression measured in the blood at screening, owing to differences in expression between tissue and blood. Therefore, if a patient is found to be IFNGS low at screening, this may not indicate the full extent of IFN activity throughout the body and could explain some apparent discrepancies in the presence or absence of improvements in clinical response in both subgroups.

Interestingly, placebo-treated patients who were IFNGS low consistently showed a higher treatment response compared with IFNGS-high patients, which could have resulted in the placebo response hiding the benefit of anifrolumab in these analyses of patients in this subgroup. This high placebo response becomes more apparent when using more sensitive disease measures. There are a number of potential explanations for this high placebo response, including a better response to standard therapy in IFNGS-low than in IFNGS-high patients, differences in baseline disease severity between these subgroups, and/or differences in the underlying mechanism of disease.

In conclusion, pooled data from the TULIP trials demonstrate an association of anifrolumab treatment with improvements in rash. Using measures of different stringency and sensitivity, the inventors show that more stringent measures (SLEDAI-2K endpoint), which require complete resolution of either rash, were largely driven by the subset of patients who were IFNGS high. Conversely, more sensitive measures are able to detect a subtle but clinically important benefit of anifrolumab treatment in small sample sizes of IFNGS-low patients. The data further support the use of anifrolumab to treat CLE.

### 11. EXAMPLE 6: SUBCUTANEOUS ADMINISTRATION OF ANIFROLUMAB

### 11.1. Phase I Study MI-CP180 of IV Anifrolumab in Patients with SSc

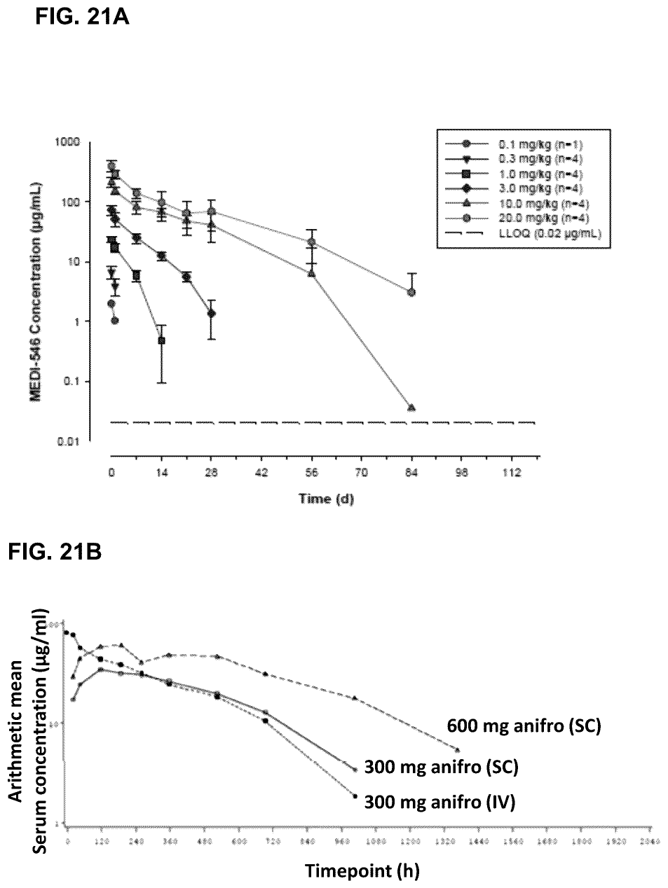

Mean anifrolumab serum concentrations after a single-dose administration based on body weight are presented in FIG. 21A. After a single-dose administration, anifrolumab exhibited nonlinear-linear PK at lower dose levels (<10.0 mg/kg) in both IFNGS high and IFNGS low patients. A dose-proportional increase in Cₘₐₓ was observed, but an increase in AUC was more than dose proportional between 0.1 and 10.0 mg/kg. Anifrolumab t1/2 was more prolonged in higher dose cohorts. At the highest dose level investigated (20.0 mg/kg), the terminal t1/2 was approximately 12 days.

### 11.2. Phase I of IV and SC Anifrolumab in Healthy Volunteers (Study 06)

In this Phase I randomized, placebo-controlled study, 30 healthy adults were assigned to three treatment cohorts (anifrolumab 300 mg SC (n=6), anifrolumab 300 mg intravenous (n=6), anifrolumab 600 mg SC (n=6)) and placebo (n=4/cohort). After SC administration, exposure to anifrolumab increased dose proportionally from 300 mg to 600 mg based on area under the serum concentration-time curve. Arithmetic mean serum anifrolumab concentration-time profiles following single IV and SC administration are shown in FIG. 21B. As reported in Tummala et al. 2018⁷, which is incorporated herein by reference in its entirety, this study estimated the bioavailability to anifrolumab in healthy volunteers to be 87% of the intravenous exposure.

### 11.3. Phase II of SC Anifrolumab in SLE Patients (Study 08)

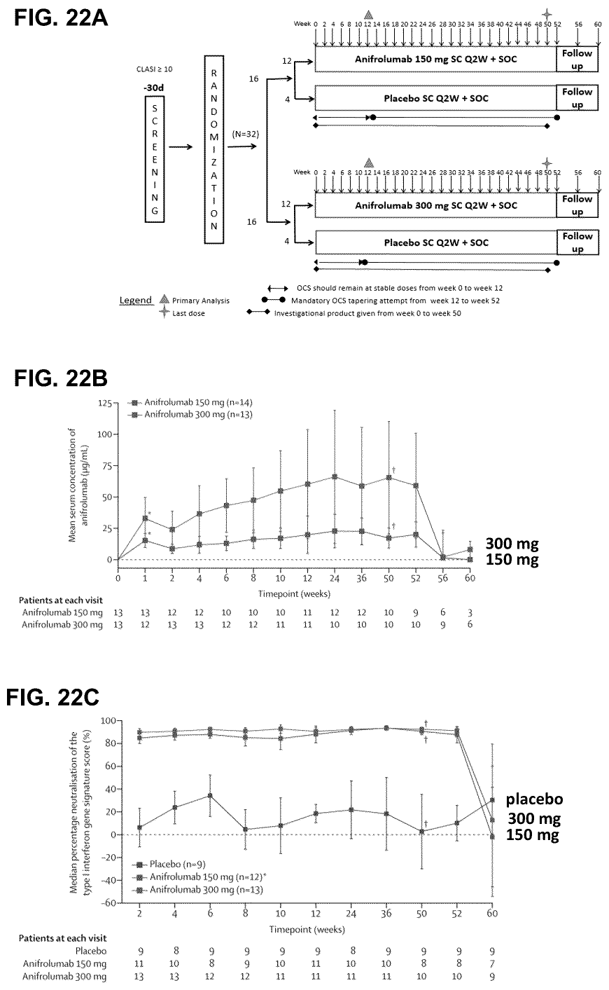

This study was designed to characterize the pharmacokinetics and pharmacodynamics of subcutaneously administered anifrolumab (FIG. 22A).

The study explored the clinical pharmacology, safety, and exploratory efficacy of subcutaneous anifrolumab. Pharmacokinetics in Study 08 were consistent with the high bioavailability in Study 06 (healthy volunteers) and high CL in IFNGS high patients with SLE. Anifrolumab, administered subcutaneously every 2 weeks to patients with SLE and moderate-to-severe skin manifestations had non-linear pharmacokinetics that were more than dose proportional, and neutralized the type I interferon gene signature in a dose-dependent manner (FIG. 22B and FIG. 22C). In particular, 150 mg or 300 mg of subcutaneous anifrolumab administered every 2 weeks for 50 weeks had non-linear pharmacokinetics, whereby C_{trough} concentrations were more than dose proportional. The number of adverse events with subcutaneous anifrolumab was similar to the numbers observed following intravenous administration in larger studies of patients with SLE.

The results of Study 08 are fully described in Bruce et al.¹⁴, which is incorporated herein by reference in its entirety.

Study 08 was limited by small samples sizes, and no conclusions could be drawn about the biological effects of the study drug (e.g., on complement C3 or C4 concentrations) or its clinical efficacy (e.g., on CLASI score). The inclusion of only patients with high type I interferon gene signatures and active skin disease also limited the generalizability of the study to patients with similar disease characteristics. The study was further limited by the increasing frequency of missing values with time.

### 11.4. Conclusion

The PK of anifrolumab consistently exhibited target mediated drug disposition where the concentrations or exposures decreased more than dose-proportional at lower dose levels. High bioavailability of anifrolumab administered via SC injection was observed in Study 06 (healthy volunteers); the ratio of the AUC of anifrolumab SC to anifrolumab IV under 300 mg was approximately 87%.

### 12. EXAMPLE 7: DETERMINATION OF THE OPTIMAL SUBCUTANEOUS UNIT DOSE

### 12.1. Aim

In order to detect an optimal dosage regimen for subcutaneous administration of anifrolumab, the inventors developed a population PK and a PK/PD model, designed to utilize existing human clinical trial. The PK data from phase III Studies 04 and 05 and phase II Study 1013 were used to assist the development of the population PK model.

An initial goal of the inventors was to detect a subcutaneous dose providing an equivalent exposure as a standard 300 mg IV (Q4W) dose, while concomitantly allowing more regular dosing that could be provided in a lower volume. This was based on the understanding that 300 mg IV Q4W provides optimal clinical PK profiles and clinical efficacy (e.g. in terms of achieving BICLA response) as reported e.g. in Furie et. al. 2017¹⁰ which is incorporated herein by reference in its entirety, and summarized in Examples 3 and 4.

### 12.2. Results

### 12.2.1. Initial Selection of the Subcutaneous Dose for Anifrolumab

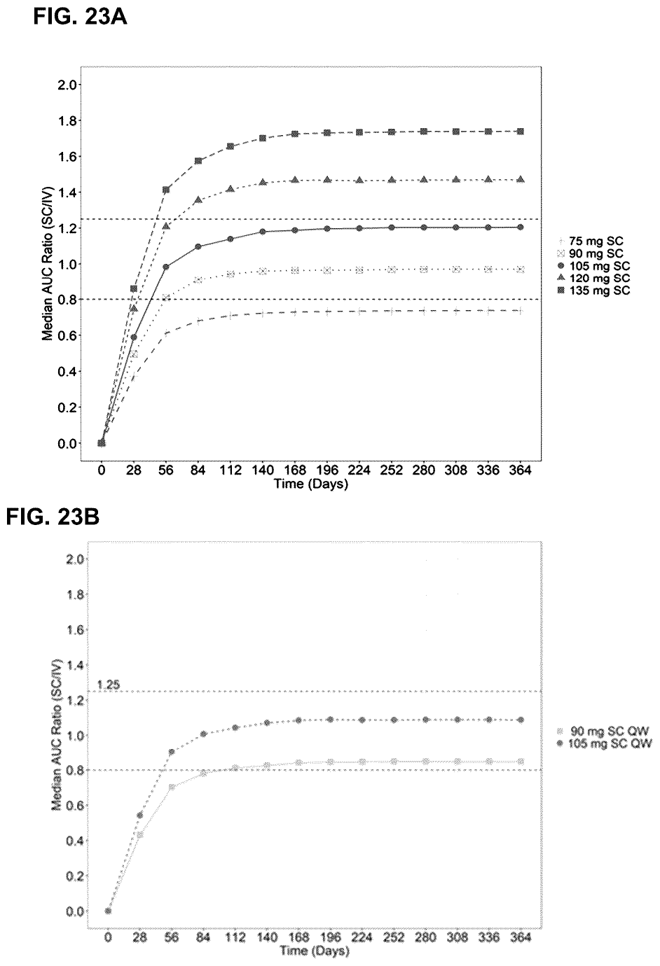

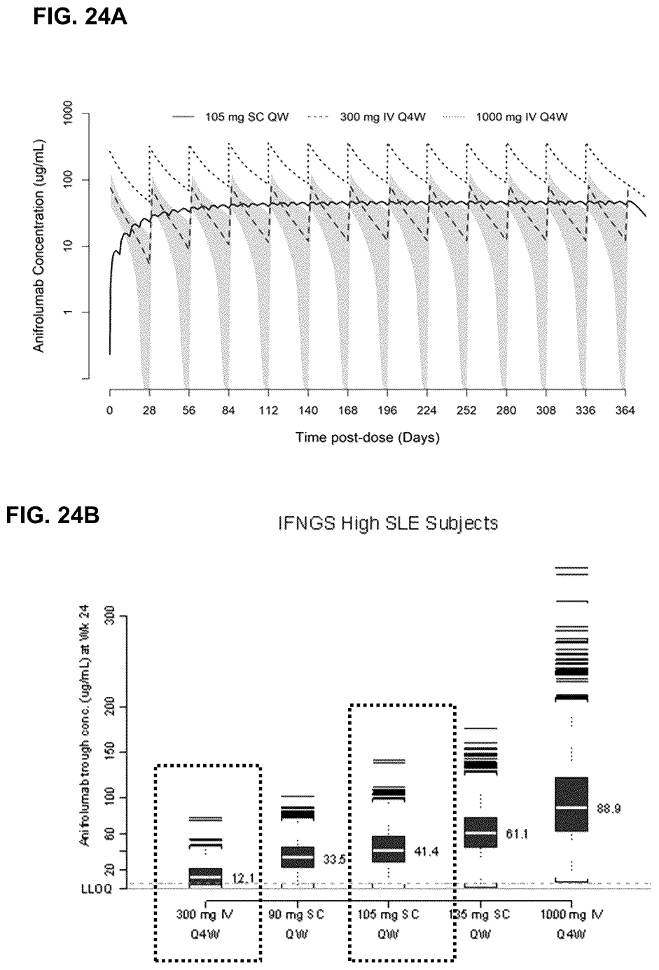

In an initial analysis, the inventors determined specific dosage regimens predicted to provide equivalent exposure to that achievable with 300 mg Q4W IV. A dosage regimen of 105 mg subcutaneous weekly (QW) was initially found to provide an AUC ratio close to (or slightly greater than) 1 (FIG. 23A), even where projected bioavailability was reduced by ˜7% relative to that reported in Tummal et. al. 2018⁷ (incorporated herein by reference in its entirety) to account for inter-individual variance in bioavailability (FIG. 23B). 105 mg subcutaneous QW appeared to provide comparable or improved median trough concentrations and IFNGS suppression as the comparative 300 Q4W mg IV dose (FIG. 24A and FIG. 24B). From these initial analyses it appeared that SC 105 mg QW dose of anifrolumab should be selected as equivalent to a 300 mg Q4W and thus as having the optimal efficacy/risk profile for the treatment of SLE patients. Importantly these analyses assumed that the 300 mg IV dose was close to the plateau of the dose response curve for anifrolumab.

### 12.2.2. Amended Selection of the Subcutaneous Dose for Anifrolumab

The inventors therefore first considered 105 mg QW to be the optimal SC dose of anifrolumab for the treatment of type I IFN mediated disease based on the data available from the MUSE study, Study 06 and Study 08. However, to confirm the selection of the 105 mg SC dose, the inventors conducted further analysis of the data from the TULIP I (Study 04) and TULIP II (Study 05) clinical trials.

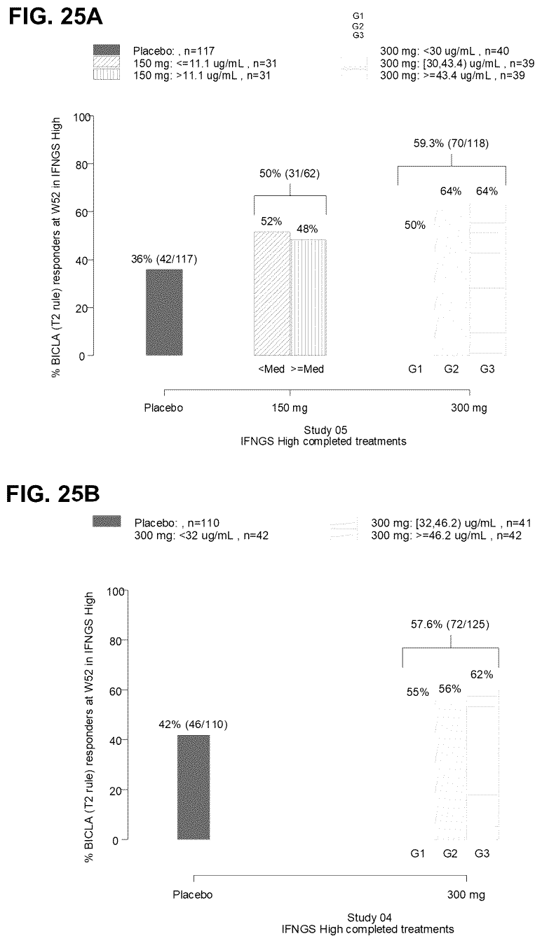

Using the additional data, a positive-exposure-BICLA relationship in IFNGS high patients was demonstrated. Surprisingly, this relationship was observed even within the 300 mg IV Q4W group (FIG. 25A and FIG. 25B). BICLA response within the 300 mg IV Q4W patient group was therefore variable. Logistic regression of the week 52 BILCA response in patients confirmed that PK exposure was a significant covariate in both TULIP I and TULIP II. Cₐᵥₑ was found to be statistically significant in both the analysis of all-comers and IFNGS high completed the treatments in both TULIP I and TULIP II independently and pooled TULIP I and TULIP II analysis. Exposure-response demonstrating higher Cave were correlated with higher BICLA and SRI(4) in pooled data from the TULIP I and TULIP II studies. In other words, there was exposure-dependent variability in response to anifrolumab within SLE patients administered 300 mg Q4W IV (FIG. 25A and FIG. 25B).

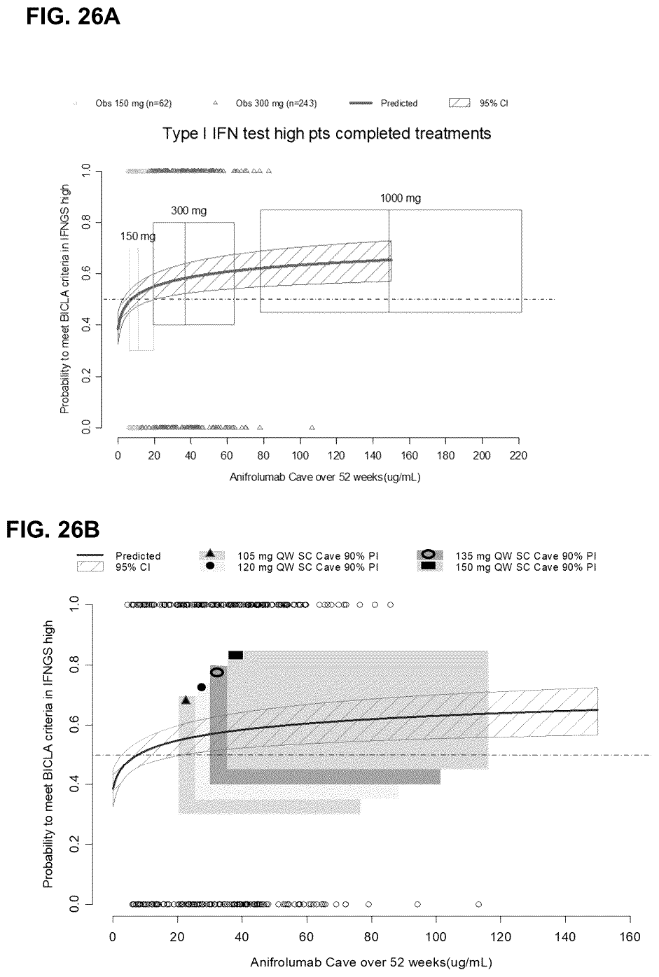

Surprisingly, the 300 mg IV Q4W dose was thus found to reside on the onset of the plateau of exposure response, whilst the suboptimal 150 mg IV dose resided in the step region of the exposure-response curve (FIG. 26A). As a consequence of these analyses, the inventors determined that a 105 mg QW subcutaneous dose (previously considered equivalent to a 300 mg IV Q4W dose) would not provide the optimal balance of efficacy and safety in SLE patients. The inventors thus determined to select another dose for SC administration that would mitigate the impact of variability in response a population of SLE patients.

In summary, from initial analysis, it appeared that administration of a subcutaneous dose of 105 mg QW anifrolumab would achieve at least a similar efficacy as 300 mg IV Q4W. However, surprisingly, following further analysis by the inventors of newly available data from further studies, it was found that the concentration of this weekly (QW) dose could be increased without reaching a maximum threshold in terms of bioavailability and efficacy. In other words, the QW dose could be increased beyond 105 mg to provide even greater blood plasma concentrations and IFNGS suppression, and to mitigate the observed variability in response in SLE patients. A dose of 105 mg would therefore be sub-optimal.

The surprising additional dose-response curve data were further validated by demonstrating that the probability of meeting a relevant BICLA response (in IFNGS high patients) was increased for weekly subcutaneous administration with concentrations higher than the 105 mg dose (Table 12-1: SC Efficacy Projection assuming no dose delays/interruptions.). These data demonstrate the unexpected position of the dose-response plateau (e.g. under subcutaneous administration), which shifts to the right for doses increasing above 105 mg (FIG. 26B), showing the maximal BICLA response is in fact achievable with a dose of greater than 105 mg and that a higher dose would be preferable (Table 12-1).

TABLE 12-1
SC Efficacy Projection assuming no dose delays/interruptions.

| | 90 mg | 105 mg | 120 mg | 135 mg | 150 mg |
|---|---|---|---|---|---|
| | SC QW | SC QW | SC QW | SC QW | SC QW |
| Equivalent IV dose | ~300 mg IV Q4W | | ~<400 mg | ~<450 mg | <500 mg |
| | | | IV Q4W | IV Q4W | IV Q4W |
| | | | | | ~300 mg |
| | | | | | SC Q2W |
| Median Cave ratio | 0.92 | 1.14 | 1.36 | 1.59 | 1.81 |
| to 300 mg IV | | | | | |
| % exceeded 95^{th} | 3.3% | 9.4% | 20.1% | 33.5% | 48.9% |
| percentile of | | | | | |
| 300 mg IV | | | | | |
| % overlapped with | 0.3% | 1.8% | 5% | 11% | 21% |
| ≥5^{th} percentile | | | | | |
| of 1000 mg IV | | | | | |
| % IFNGS high pts | ~86% | ~94% | ~98% | ~99% | ~100% |
| with 55% chance of | | | | | |
| BICLA response | | | | | |
| % IFNGS high pts | ~10% | ~23% | ~38% | ~55% | ~68% |
| with 60% chance of | | | | | |
| BICLA response | | | | | |

### 12.2.3. The Bioavailability of Anifrolumab is Highly Variable

Upon further investigation as to the bioavailability of anifrolumab, the inventors elucidated that a surprisingly high level of variability in anifrolumab bioavailability subsequent to subcutaneous administration may exist amongst different patients. The high level of variability in anifrolumab bioavailability was not appreciated in previous studies reporting >80% bioavailability for subcutaneous administration (see Example 3)⁷. The bioavailability (F1) of anifrolumab in Study 08 (SLE patients, SC) was found to be 81% in healthy volunteers using the population PK model (Table 12-2).

TABLE 12-2
Anifrolumab bioavailability based on healthy volunteers

| | Final IV SLE model | Study of 06 HVs |
|---|---|---|
| | (including 6 subjects | (300 mg IV:6, 300 mg |
| Parameters ± SE | from 06 IV arm) | SC: 6, 600 mg SC: 6) |
| CL (IFNGS high) | 0.193 L/day | — |
| CL (IFNGS low/HVs) | 0.153 L/day | 0.146 ± 0.036 L/day |
| IIV:CL | 0.109 (CV: 33.1%) | 0.0431 (CV: 20.8%) |
| F1 | — | 0.812 ± 0.12 |
| Ka | — | 0.274 ± 0.124/day |
| IIV: Ka | — | 0.221 (CV: 47%) |

The bioavailability of a typical monoclonal antibody via subcutaneous injection ranged from 52-80%¹⁵. The inventors conducted external validation of Study 08, Ph2 SC in SLE, using a PPK model developed with healthy volunteers and SLE patients from IV studies to determine the bioavailability in SLE population.

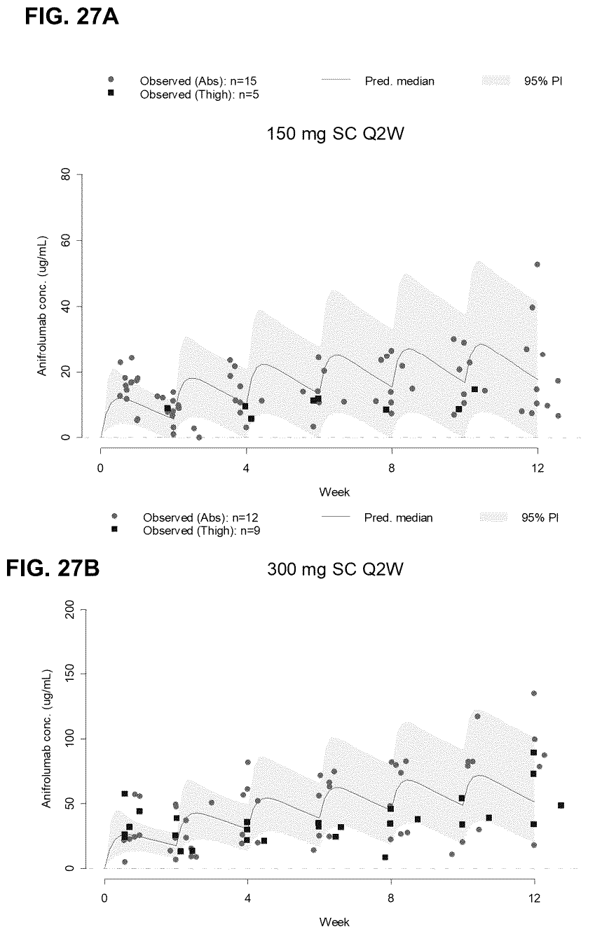

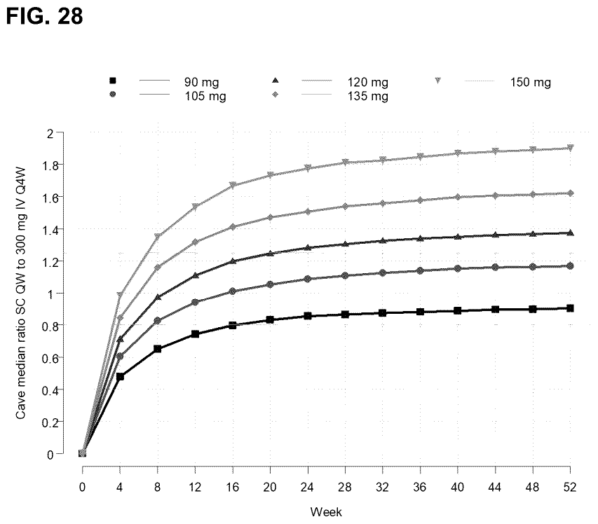

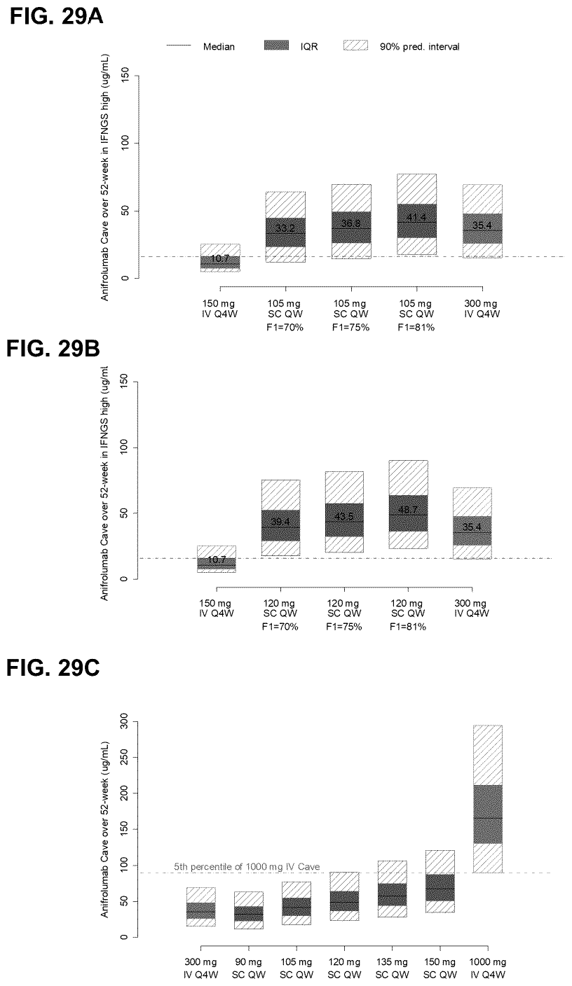

In-depth analysis of the data from Study 08 revealed that bioavailability was affected by SC administration site. In particular, when the bioavailability of 300 mg at the abdomen was estimated versus IV, the bioavailability (F1) was estimated to be 85.4% compared to 81% when the sites of injection was not taken into consideration. As such, C_{troughs} following injection at thigh trended downward compared to injection at abdomen (FIG. 27A and FIG. 27B). As such, it was surprisingly concluded that bioavailability may, in fact, be as low as 70%, taking into account variability due to injection site and the higher variability in bioavailability for SLE patients compared to healthy volunteers. Importantly, if a bioavailability (F1) of 81-87% was assumed, 105 mg was initially projected to provide a comparable Cₐᵥₑ to that of 300 mg IV (FIG. 28). By contrast, when the estimated bioavailability was reduced to ˜70% or less, the median Cₐᵥₑ of the 105 ma QW subcutaneous dose fell to below 1 (FIG. 29A. FIG29B and Table 12-3).

TABLE 12-3
Anifrolumab bioavailability

| | 90 mg | 105 mg | 120 mg | 135 mg | 150 mg |
|---|---|---|---|---|---|
| Bioavailability | SC QW | SC QW | SC QW | SC QW | SC QW |
| 82% | 0.92 | 1.14 | 1.36 | 1.59 | 1.81 |
| ~70% | 0.73 | 0.92 | 1.11 | 1.31 | 1.49 |
| ~60% | 0.57 | 0.73 | 0.89 | 1.06 | 1.22 |

Values = median Cₐᵥₑ to 300 mg IV;
SC = subcutaneous

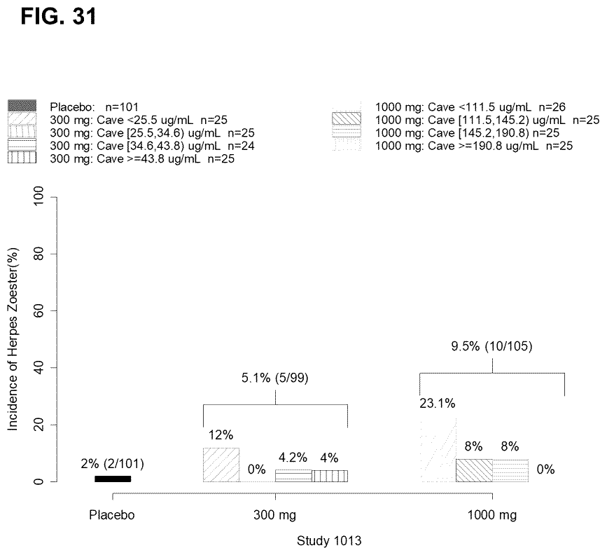

Furthermore, there was an undesirable 30% overlap in Cave between 105 mg SC QW and the suboptimal IV dose, 150 mg Q4W versus the only 16% overlap observed when the bioavailability was assumed to be 81% (FIG. 29A). However, when a SC 120 mg dose was used, the Cₐᵥₑ overlap with the 150 mg IV dose was less than the overlap with the optimal IV dose of 300 mg IV, even when a low bioavailability of 70% was assumed (FIG. 29B). Furthermore, the 120 mg SC QW dose had minimal overlap with the undesirable 1000 mg IV dose (FIG. 29C), at which the risk of herpes zoster infection is increased (FIG. 31). A 150 mg SC QW dose had an undesirable overlap with the 1000 mg IV Q4W dose. Even more surprisingly, a SC dose of 120 mg or more was projected to have better PD suppression (Table 12-4) than the assumed optimal 300 mg IV dose (Table 12-5).

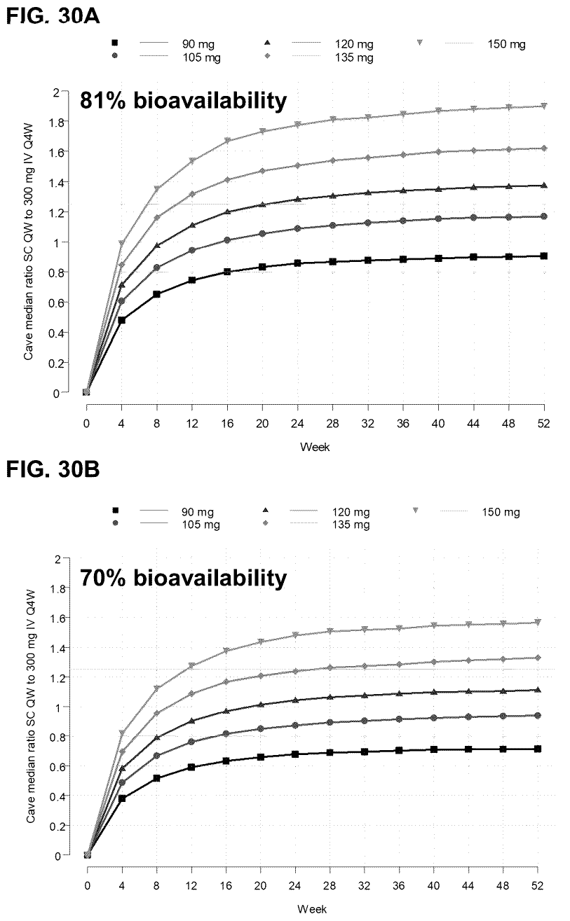

Selection of a dose higher than 105 mg, preferably 120 mg or higher, therefore optimizes the exposure-response by minimizing the impact of the variability of the onset of response and bioavailability in patients with SLE (Table 12-4, FIG. 30A and FIG. 30B). A SC dose of below 150 mg QW is also desirable to reduce the risk of herpes zoster infection.

TABLE 12-4
Calculated % PD suppression at week 24, SC dose

| SC | WK24 Suppression (%) | | |
|---|---|---|---|
| Dose (mg) | 75% | 80% | 90% |
| 90 | 89.0 | 84.6 | 63.8 |
| 105 | 92.9 | 89.8 | 69.2 |
| 120 | 94.8 | 91.9 | 74.2 |
| 135 | 96.0 | 93.9 | 75.8 |
| 150 | 96.5 | 94.6 | 80.2 |

TABLE 12-5
Calculated % PD suppression at week 24, IV dose

| IV | WK24 Suppression (%) | | |
|---|---|---|---|
| Dose (mg) | 75% | 80% | 90% |
| 300 | 74.2 | 68.3 | 42.5 |
| 400 | 82.9 | 77.9 | 54.7 |
| 450 | 85.9 | 80.8 | 56.4 |
| 500 | 88.7 | 84.8 | 62.5 |
| 600 | 92.7 | 88.8 | 68.9 |
| 1000 | 96.9 | 94.5 | 80.2 |

Doses of 120 mg and 135 mg QW particularly provide reasonable benefit-risk profiles. At doses at 150 mg QW or above, there is an increase in safety risk e.g. an increase in the risk of herpes zoster in patients, given that a SC dose of 150 mg QW is equivalent to a 1000 mg IV Q4W (FIG. 29C, FIG. 31). A subcutaneous dose of less than 150 mg QW and more than 105 mg QW was therefore determined as the preferred dose. A subcutaneous dose of less than 150 mg QW and less or equal to 135 mg was determined as the more preferred dose. A subcutaneous dose of 120 mg was determined as optimal dose.

To summarize, the inventors have surprisingly found that the optimal subcutaneous dose of anifrolumab may first appear to be 105 mg QW given the preliminary data that was previously available (FIG. 12). However, further data and analyses surprisingly revealed that a dose of 105 mg QW or lower would under-dose a significant proportion of patients (FIG. 26B, Table 12-3). Thus, a particularly advantageous dosing regimen demonstrated by the inventors was doses higher than 105 mg QW. A particularly optimal dose was determined to be 120 mg subcutaneous QW, which is equivalent to approximately 400 mg IV Q4W, depending on estimated bioavailability. The optimal SC dose is therefore surprisingly >30% higher than what would be considered optimal based solely on a comparison with 300 mg IV Q4W and the previously understood bioavailability of anifrolumab.

The inventors have thus surprisingly demonstrated that a dose of greater than 105 mg SC QW and less than 150 mg SC QW, and in particular a dose of 120 mg QW (a) maximizes efficacy whilst maintaining an acceptable safety profile, (b) mitigates the impact of variability in bioavailability and (c) mitigates the impact of variability in the onset of response. Thus, dosing at greater than 105 mg QW advantageously accounts for the variance in bioavailability, leading to improved therapeutic outcome. A dose of less than 150 mg QW mitigates the risk of herpes zoster infection.

Pharmacokinetic data in healthy volunteers (study 06 [IV arm only]) and in patients with SLE (Studies 1013, 02, 04, and 05) were also pooled to evaluate the impacts of covariates, such as demographics and renal/liver function tests, on PK exposure. Higher body weight and type I IFN test high patients were found to have significantly higher clearance (CL) and lower concentrations. However, surprisingly there was no clinically relevant impact of these covariates on efficacy and safety. Surprisingly, other covariates pertaining to specific populations evaluated in population PK modeling were not found to be significant including race/ethnicity/region, age, gender, renal/hepatic function tests, standard of care therapy (e.g., OCS, anti-malarial, azathioprine, methotrexate, mycophenolate mofetil, mycophenolic acid, mizoribine, and NSAIDs), and commonly used medications in SLE patients (ACE inhibitors and HMG-COA reductase inhibitors).

### 12.3. Conclusion

The present inventors have demonstrated that an anifrolumab dose of <150 mg Q and >105 mg QW will provide at least similar or even a higher Cₐᵥₑ over 52 weeks to that of 300 mg IV Q4W. A 120 mg SC QW dose will particularly provide an efficacy at least equivalent to that demonstrated for a 300 mg IV Q4W dose. It is further plausibly demonstrated that a 120 mg SC QW dose will provide an efficacy greater than that demonstrated for a 300 mg IV Q4W dose.

A dosing regimen of 900 mg anifrolumab IV Q4W for 6 doses followed by 120 mg anifrolumab SC QW was thus selected based on a combination of PK/PD data and modelling of data from the Phase II LN study (Study 7, see Section Error! Reference source not found . . . and Error! Reference source not found.) and knowledge from the anifrolumab IV and SC clinical program in SLE as described Section 12.2.

Study 07, assessed 2 dosing regimens: a basic regimen using the proposed dose for SLE patients (300 mg IV Q4W) and an intensified regimen, which commenced with 3 doses of 900 mg IV Q4W followed by 300 mg IV Q4W for the remainder of the study. The intensified regimen showed results suggesting a greater treatment benefit over the basic regimen.

However, the exposure in the initial phase 300 mg IV Q4W of both regimens was suboptimal when compared with the 300 mg IV Q4W dose in SLE without active renal disease. Therefore, a more intensive dosing regimen was selected, with an initial dose of 900 mg IV Q4W for 6 doses, followed by 120 mg SC QW. This regimen will provide sustained anifrolumab exposure/PD suppression and improved UPCR outcome compared with the previously evaluated dose regimens in study 07. The 120 mg SC QW dose will provide at least similar or non-inferior exposure and PD suppression to that of 300 mg IV Q4W in patients with SLE.

The IV route of administration has primarily been used in the anifrolumab clinical program in SLE without active renal disease, but a more convenient SC route of administration is also being developed. Both the IV and SC routes of administration have been shown to be safe and well tolerated in the anifrolumab clinical development program in patients with SLE (IV and SC) and in LN (IV). The SC route of administration using aPFS for anifrolumab is expected to provide increased convenience and dosing flexibility and reduced exposure to infection risk related to clinic visits for dosing (including but not limited to influenza or COVID-19) for patients and/or caregivers and to improve treatment accessibility and compliance compared to IV dosing.

### 13. EXAMPLE 8: INJECTION DEVICE

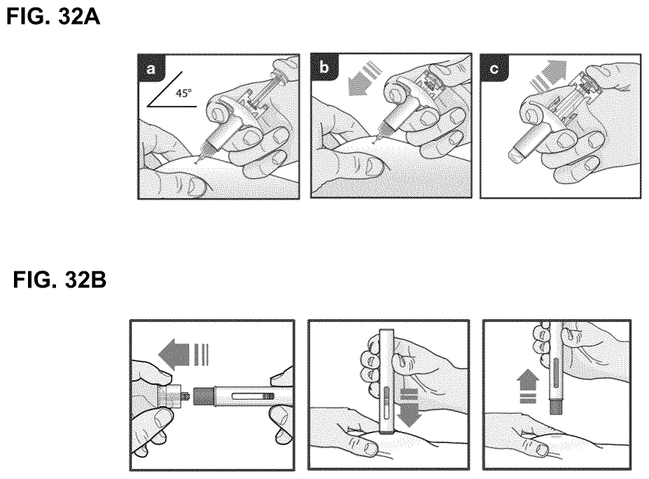

Anifrolumab is administered by an injection device [1] [9] such as a prefilled syringe (PFS) (FIG. 32A) or an autoinjector (AI) (FIG. 32B).

### 13.1. Autoinjector

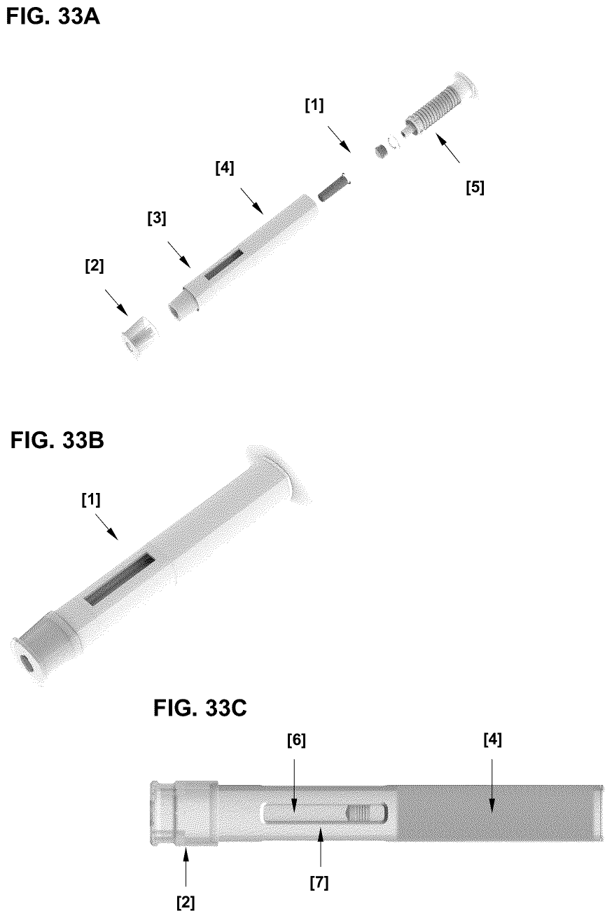

Anifrolumab may be administered by an autoinjector [1]. The autoinjector is shown in exploded view (FIG. 33A) and in an assembled form (FIG. 33B). A label [4] is wrapped around and attached to the autoinjector [1] (FIG. 33C). The autoinjector has an autoinjector housing [3], cap and cap remover [2] and drive unit [5]. The liquid anifrolumab formulation unit dose [6] is contained in the autoinjector housing [3]. The unit dose [6] can be viewed through the viewing window [7].

### 13.2. Accessorized Pre-Frilled Syringe

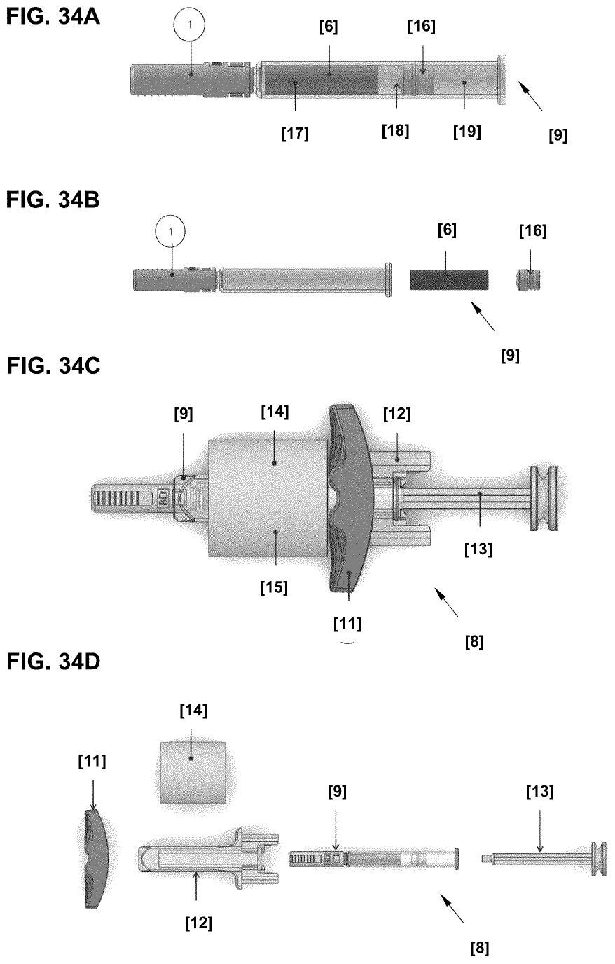

Anifrolumab may be administered by accessorized pre-filled syringe (APFS) [8]. The APFS [8] includes the unit dose of anifrolumab [6] contained in a primary container [9] shown in an assembled state in FIG. 34A and in an exploded view in FIG. 34B. The primary container [9] has a plunger stopper [16]. The primary container has a nominal fill volume [17] of 0.8 ml but may contain slightly more than 0.8 ml. The remainder of the space in the primary container [9] is taken up by an air bubble [18]. The air bubble [18] may have a size of 3-5 mm, optionally, 4 mm. The primary container [9] has a defined stopper position [19].

The accessorized pre-filled syringe (APFS) primary container [9] is provided in a PFS assembly [8] including a needle guard [12], a finger flange [11] and a plunger rod [13]. A label [14] is provided with the

primary container [9] in the PFS assembly [8]. The label [14] is wrapped around the syringe [9] in the label placement position [15].

### 13.3. Packaging

The injection device [1] [8] is provided in a kit [20] (FIG.). A label [4] [14] is provided with the APFS or autoinjector in the packaging. The label includes instruction for the use of the injection device [1], [8]. The packaging includes a tamper seal.

### REFERENCES

All publications mentioned in the specification are herein incorporated by reference.
(1) Shi, H.; Gudjonsson, J. E.; Kahlenberg, J. M. Treatment of Cutaneous Lupus Erythematosus: Current Approaches and Future Strategies. Curr. Opin. Rheumatol. 2020, 32 (3), 208-214. https://doi.org/10.1097/BOR.0000000000000704.
(2) Jarrett, P.; Werth, V. P. A Review of Cutaneous Lupus Erythematosus: Improving Outcomes with a Multidisciplinary Approach. J. Multidiscip. Healthc. 2019, 12, 419-428. https://doi.org/10.2147/JMDH.S179623.
(3) Furie, R.; Khamashta, M.; Merrill, J. T.; Werth, V. P.; Kalunian, K.; Brohawn, P.; Illei, G. G.; Drappa, J.; Wang, L.; Yoo, S.; Investigators, for the C. S. Anifrolumab, an Anti-Interferon-α Receptor Monoclonal Antibody, in Moderate-to-Severe Systemic Lupus Erythematosus. Arthritis Rheumatol. Hoboken Nj 2017, 69 (2), 376. https://doi.org/10.1002/art.39962.
(4) Tummala, R.; Rouse, T.; Berglind, A.; Santiago, L. Safety, Tolerability and Pharmacokinetics of Subcutaneous and Intravenous Anifrolumab in Healthy Volunteers. Lupus Sci. Med. 2018, 5 (1), e000252. https://doi.org/10.1136/lupus-2017-000252.
(5) Ferguson, G. T.; Mansur, A. H.; Jacobs, J. S.; Hebert, J.; Clawson, C.; Tao, W.; Wu, Y.; Goldman, M. Assessment of an Accessorized Pre-Filled Syringe for Home-Administered Benralizumab in Severe Asthma. J. Asthma Allergy 2018, 11, 63-72. https://doi.org/10.2147/JAA.S157762.
(6) A Pilot Study Characterizing Factors in Adherence to Cutaneous Lupus Treatment—Teresa Ro, Rechelle Z. Tull, Leah A. Cardwell, Michael E. Farhangian, Farah Moustafa, Irma Richardson, Joseph L. Jorizzo, Steven Feldman, William Huang, 2018 https://journals.sagepub.com/doi/abs/10.1177/1203475418770581 (accessed Apr. 12, 2021).
(7) Albrecht, J.; Taylor, L.; Berlin, J. A.; Dulay, S.; Ang, G.; Fakharzadeh, S.; Kantor, J.; Kim, E.; Militello, G.; McGinnis, K.; Richardson, S.; Treat, J.; Vittorio, C.; Van Voorhees, A.; Werth, V. P. The CLASI (Cutaneous Lupus Erythematosus Disease Area and Severity Index): An Outcome Instrument for Cutaneous Lupus Erythematosus. J. Invest. Dermatol. 2005, 125 (5), 889-894. https://doi.org/10.1111/j.0022-202X.2005.23889.x.
(8) Klein, R. S.; Morganroth, P. A.; Werth, V. P. Cutaneous Lupus and the CLASI Instrument. Rheum. Dis. Clin. North Am. 2010, 36 (1), 33-51. https://doi.org/10.1016/j.rdc.2009.12.001.
(9) Touma, Z.; Urowitz, M.; Gladman, D. SLEDAI-2K for a 30-Day Window. Lupus 2010, 19 (1), 49-51. https://doi.org/10.1177/0961203309346505.
(10) Interferon-Inducible Gene Expression Kit As a Potential Diagnostic Test for Anifrolumab: Analytical Validation for Use in Clinical Trials. ACR Meeting Abstracts.
(11) Furie, R. A.; Morand, E. F.; Bruce, I. N.; Manzi, S.; Kalunian, K. C.; Vital, E. M.; Ford, T. L.; Gupta, R.; Hiepe, F.; Santiago, M.; Brohawn, P. Z.; Berglind, A.; Tummala, R. Type I Interferon Inhibitor Anifrolumab in Active Systemic Lupus Erythematosus (TULIP-1): A Randomised, Controlled, Phase 3 Trial. Lancet Rheumatol. 2019, 1 (4), e208-e219. https://doi.org/10.1016/S2665-9913(19)30076-1.
(12) Morand, E. F.; Furie, R.; Tanaka, Y.; Bruce, I. N.; Askanase, A. D.; Richez, C.; Bae, S.-C.; Brohawn, P. Z.; Pineda, L.; Berglind, A.; Tummala, R. Trial of Anifrolumab in Active Systemic Lupus Erythematosus. N. Engl. J. Med. 2020, 382 (3), 211-221. https://doi.org/10.1056/NEJMoa1912196.

1-42. (canceled)

43. A pharmaceutical unit dose form comprising >105 mg and ≤150 mg anifrolumab.

44. The unit dose of claim 43, wherein the unit dose comprises ≤135 mg anifrolumab.

45. The unit dose of claim 43, wherein the unit dose comprises about 120 mg anifrolumab.

46. The unit dose of claim 45, wherein the unit dose consists essentially of >105 mg and <150 mg anifrolumab.

47. The unit dose of claim 46, consisting essentially of ≤135 mg anifrolumab.

48. The unit dose of claim 47, consisting essentially of about 120 mg anifrolumab or the functional variant thereof.

49. The unit dose of claim 43, wherein the concentration of anifrolumab in the unit dose is about 150 mg/ml.

50. The unit dose of claim 43, wherein the volume of the unit dose is about 0.8 ml.

51. The unit dose for the use of claim 43, wherein the unit dose comprises a formulation of about 150 to 200 mg/ml anifrolumab, about 25 to 150 mM of lysine salt and an uncharged excipient.

52. (canceled)

53. An injection device comprising the unit dose of claim 43.

54. The injection device of claim 53, wherein the injection device is a pre-filled syringe (PFS).

55. The injection device of claim 54, wherein the injection device is an accessorized pre-filed syringe (AFPS).

56. The injection device of claim 53, wherein the injection device is an auto-injector.

57-66. (canceled)

67. A method of treating a type I IFN mediated disease in a subject in need thereof, the method comprising subcutaneously administering a dose of >105 mg and ≤150 mg anifrolumab to the subject.

68. The method of claim 67, wherein the method comprises the subcutaneously administering a dose of 120 mg anifrolumab to the subject.

69. The method of claim 67, wherein the method comprises the subcutaneously administering a dose of 120 mg anifrolumab to the subject once per week (QW).

70. The method of claim 67, wherein the type I IFN mediated disease is systemic lupus erythematosus (SLE) or cutaneous lupus erythematosus (CLE).
